(19) United States
(12) Patent Application Publication
(10) Pub. No.: US 2008/0171945 A1
(43) Pub. Date: Jul. 17, 2008

(54) APPARATUS AND METHOD FOR MEASURING HEART RATE AND OTHER PHYSIOLOGICAL DATA

(76) Inventor: James E. Dotter, Boulder, CA (US)

Correspondence Address: DUFT BORNSEN & FISHMAN, LLP, 1526 SPRUCE STREET, SUITE 302, BOULDER, CO, 80302, US

(21) Appl. No.: 11/942,934

(22) Filed: Nov. 20, 2007

Related U.S. Application Data
(60) Provisional application No. 60/884,958, filed on Jan. 15, 2007.

(51) Int. Cl. A61B 5/04; A61B 5/08

(52) U.S. Cl. 600/514; 600/519; 600/529

(57) ABSTRACT

Apparatus for measuring a user's heart rate and other physiological parameters derived from two electrodes at least one of which is in contact with the user's head. The second electrode is in contact with the user's skin. A heart rate detection circuit is coupled to the electrodes to detect the user's heart rate as electrical potential difference between the first and second electrodes. The electrodes may be integral with user apparel such as hats, headbands, helmets, eyewear, etc. The electrodes may be integral with headphone speakers in contact with the user's ears. The heart rate detection circuit may be integral with or coupled to an audio source device such as an MP3 player or a portable communication device. The detected heart rate may be presented to the user as audio signals or as visually displayed information. A third electrode may be added to improve the quality of the sensed signals.

### BACKGROUND

1. Related Applications

This application claims priority to U.S. provisional patent application Ser. No. 60/884,958 filed 15 Jan. 2007 and entitled: DEVICE AND METHOD FOR MEASURING HEART RATE AND OTHER PHYSIOLOGICAL DATA.

2. Field of the Invention

The invention relates to measurement of heart rate and other physiological data such as respiratory rate and more specifically relates to an apparatus and method for monitoring heart rate and/or respiratory rate using probe devices integral with an entertainment device usable in sporting endeavors.

3. Discussion of Related Art

Current methods for the measurement of heart rate generally fall into a few categories as described herein below.

One measurement method is acoustic. There are familiar acoustic properties of a beating heart. A stethoscope is traditionally used to amplify these sounds and present them to a caregiver. The acoustic principle may also be used in other ways, both manual and automated, at various parts of the body.

Another measurement method is an optical approach. Products in this category shine a light of a known frequency through an area of the body, such as the fingertip or earlobe, and detect the same light once it has either passed through the body or been reflected back to a photo sensor. With each heart beat, oxygen-rich blood is momentarily pushed through the capillaries in that region. This momentary increase in the oxygen content of the blood upon each heart beat changes the optical properties of the blood. As the light passes through the fingertip or earlobe, specific frequencies are absorbed to varying degrees, depending on the amount of oxygen in the blood, and are therefore not present in the returning light. The change in detected frequencies occurring once per heart beat allows for detection of individual heart beats, and thus a heart rate measurement. The degree of spectral change is used to determine the oxygen content in the blood. This method is commonly used to monitor heart rate and oxygen saturation of patients in a hospital, via products referred to as pulse-oximeters.

The same optical principle has been applied to sports and fitness, as an ear-clip optical device. This approach has been used with treadmills, exercise bikes, and other stationary types of exercise. This has been used with good results in situations where the wearer's head does not move too much or too vigorously during exercise. Traditional designs have shown an earlobe-clip which houses the light source and light sensor, with a wire connecting it to the required circuitry which is located in the exercise equipment's console. Some prior designs discuss or suggest integrating the photo-sensor and associated electronics into a headset.

Another measurement method makes use of the varying outward pressure applied against the skin by major arteries. With each heart beat, a surge of blood passes through the arteries. In an artery of sufficient size, and located near to the surface of the body, this momentary pressure can be detected by holding a pressure sensor, such as a piezo-electric (P-E) element, in place over the artery location. The P-E element is physically stretched by the momentary outward pressure of the artery during a heart-beat. As it is stretched, the altered shape of the P-E element changes its electrical characteristics—e.g., a change in its resistance to a current passing through it. Changes in the resistance of the P-E are then detected by appropriate circuitry, and used to identify heart beats and thus heart rate. Suitable surface arteries and sensing devices are well known in the art and include sensing at the wearer's wrist, the temple, the inner ear, or the bridge of the nose.

Electrocardiogram (ECG or EKG) measurement is used for medical diagnostic purposes related to ailments of the heart. This is based upon measurement of a voltage potential between electrodes positioned at contact points on the torso, arms or legs. This method is principally concerned with providing a detailed waveform used in cardiology to detect various heart malfunctions. Measurement of heart rate is a natural by-product of the more complex waveform detection and presentation of ECG/EKG.

In cardiology, a “lead” is defined as a precise positioning of two or more electrodes in contact with the skin, across which the voltage is measured. By attaching electrodes to multiple locations and measuring multiple combinations of these electrodes, cardiologists define multiple leads, or views of the heart's electrical activity. Standard combinations of leads provide diagnostic information, with 3, 12, and 14 lead ECG studies being common. ECG monitors are generally stationary, but portable versions called Holter monitors also exist to facilitate the capture of infrequent irregularities while the patient goes about a normal day.

Another method for heart rate measurement, found in many products designed for sports and fitness training, measures the voltage potential across two areas of the chest, on the left and right sides of the sternum. Heart rate monitors for this purpose consist of a strap placed around the chest of the user, with electrical contact areas (electrodes) on the left and right side of the front of the chest in contact with the skin. The elastic band on which the electrodes are mounted hold the apparatus in place. While these devices measure a voltage across the chest similar to an ECG, these devices are designed to provide only heart rate and not the full diagnostic waveform produced by ECG equipment. For this reason, the electrodes in a chest strap are not required to be as precisely positioned as ECG leads. Although they must be positioned over the ribcage with the electrodes placed properly (e.g., one on each side of the wearer's chest), a degree of flexibility in the placement of the strap makes it suitable for use by the general public rather than health care providers. These devices typically transmit the detected heart beats wirelessly to a wrist watch or other display device.

For sports and fitness training, the chest strap approach has many advantages over the other methods. They are more comfortable and less expensive than an ECG and more suitable for sports activities. The placement of the chest strap generally does not impede physical activity to the same degree as other sensor placements (such as fingertip placement) and the chest strap is more comfortable than an earlobe clip.

More importantly, optical, acoustic and arterial pressure sensing devices have all been found to be impractical for many sports activities due to a tendency to become unreliable when the wearer moves about during the course of exercise. In all three of these techniques, even a small amount of vibration of the input sensor can introduce ‘motion artifacts’ into the data that can obliterate the desired signal. Also, these three techniques require a more precise positioning of the sensor. When the user's activity moves the sensor away from the precise required position, it fails to operate properly. The chest strap approach has been found to be more forgiving of motion and of positioning error, thus less susceptible to these faults than acoustic, optical or pressure sensing methods.

For all of the above reasons, heart rate monitoring using the chest strap method has become increasingly popular for sports and fitness training as well as for some other activities such as relaxation training, stress relief and meditation in which heart rate as a bio-feedback item has been found useful. During this time, the chest strap has remained in much the same form, as a practical means of obtaining a continuous, accurate heart rate reading for these largely non-medical purposes.

However, for many users, the chest strap may chafe causing discomfort. Many users find them awkward to put on, uncomfortable to wear, and bothersome to keep handy. In addition, they can be restrictive of good chest expansion and thus restrict full breathing during exercise. For wearers with slender ribs and torsos, the chest strap can slip down out of the proper position and cease to function properly. Stretched across the chest, they are perceived by some as unmanly, or unwomanly, or as interfering with tan lines or undergarments.

In view of the above discussion, there is an ongoing need for an improved structure and method for heart rate monitoring (and other physiological parameter monitoring) useful for sporting endeavors and other applications.

### SUMMARY

The present invention solves the above and other problems, thereby advancing the state of the useful arts, by providing an improved monitor for monitoring heart rate and other physiological parameters of the wearer. In view of the foregoing, a broad objective hereof is to provide an improved heart rate monitoring device, offering continuous, accurate heart rate measurement which is portable and comfortable to wear during normal daily life, and yet is reliable for use during physical activity. Another objective is to provide all of the favorable and positive characteristics of a voltage measurement method of heart rate detection such as used in chest-straps, but using a more comfortable or convenient structure to position the monitor on the user. The goal is to provide a reliable tool for applications such as sports and fitness training, meditation, stress-relief therapy or other bio-feedback uses, where continuous, accurate heart rate measurement is desired while the user is in motion, and where comfort and convenience are paramount and style may be a factor.

### BRIEF DESCRIPTION OF THE DRAWINGS

FIG. 1 displays elements of a system in an exemplary embodiment.

FIG. 2 displays elements of a system in another exemplary embodiment.

FIG. 3 displays elements of a system in another exemplary embodiment.

FIG. 4 displays elements of a system in another exemplary embodiment.

FIG. 5 details the electrical connections between various components of an exemplary system such as in FIGS. 1 through 4.

FIG. 6 details electrical connections in an exemplary system of an embodiment where a third electrode is present.

FIG. 7 displays a detailed description of the analytical processes involved in processing exemplary monitored signals.

FIG. 8 displays a block diagram of various exemplary components of an exemplary embodiment.

FIG. 9 details the electrical connections between various components of an exemplary system such as in FIGS. 1 through 4.

FIG. 10 shows a typical heart beat waveform, as recorded by a standard ECG machine, with standard elements P, Q, R, S and T of the ECG waveform labeled.

FIG. 11 shows exemplary electrical signals recorded by a standard ECG machine with only two electrodes positioned one on each side of the wearer's chest as presently known in the art such as for a chest strap monitor.

FIGS. 12 through 23 show electrical signals recorded from various embodiments hereof, as measured by a standard ECG machine specially configured to use electrodes in accordance with features and aspects hereof.

FIGS. 24 and 25 detail electrical connections in exemplary systems of embodiments where a third electrode is present and wiring for the electrodes is in common with wiring for the audio source.

### DETAILED DESCRIPTION OF THE DRAWINGS

While not described as a medical diagnostic tool, features and aspects hereof build upon techniques and nomenclatures of standard electrocardiography and thus the description herein shares some of that terminology. This discussion expands upon current practice in electrocardiography by using previously undisclosed electrode locations, and therefore defining new ‘leads’. Features and aspects hereof take advantage of certain characteristics of the signals derived from these new leads for the purpose of detecting and precisely timing heart beats.

To accomplish the above stated objectives, features and aspects hereof provide a heart rate monitoring system comprising, in part, at least two electrically conductive surfaces (herein called ‘electrodes’) adapted to fit against the skin of a wearer. At least one of the electrodes is designed to be placed in contact with the skin on one side of the head. A second electrode is designed to be placed in contact with the skin of the head, arm, torso or leg. Wires connect these electrodes to circuitry capable of measuring the electrical voltage potential between the electrodes and to detect patterns therein corresponding to individual heart-beats. Features and aspects hereof also comprise circuitry and connections to determine a heart rate and other derived data from the individual heart-beats, and to report these data to the user in various ways.

Voltage potential measurement has been used to detect heart rate in both ECG and chest-strap methods, however nothing in presently practiced techniques or devices describes an apparatus or method in which the voltage potential is measured by positioning at least one of the two required electrodes at a location on the side of the head. Indeed, nothing in the prior techniques or structures suggests that it would be possible to measure cardiac-related voltages at any locations other than on the torso or limbs. One aspect hereof therefore is the placement of at least one of the electrodes on the side of the head, including locations in, on or around the ear, or on the temple, for the purpose of measuring voltage changes derived from cardiac neuromuscular activity. Features and aspects hereof are presented using nomenclature of new ECG ‘leads’, or views of the heart. These leads have not been described in prior techniques and structures and are made possible by a novel positioning of electrodes.

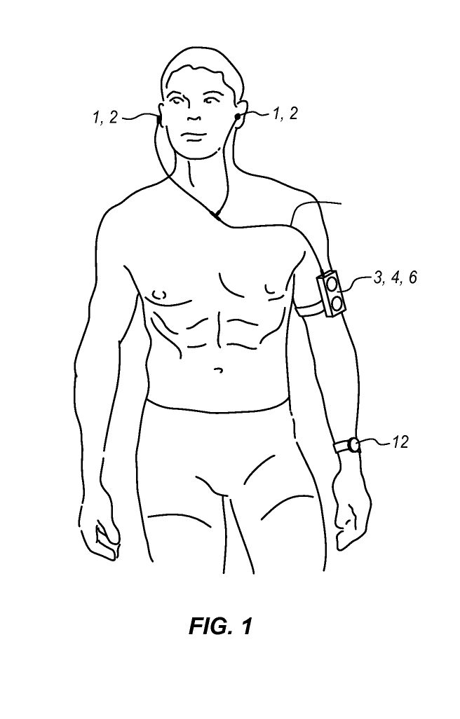

One exemplary arrangement of the above described electrodes is to have at least one electrode positioned to be in contact with the skin of the head, including the ear, and a second electrode positioned to be in contact either with the skin of the arm at the bicep or wrist, or else with the skin of the torso at the waist, as shown in FIGS. 1, 2, and 3. This arrangement offers the benefit of practicality and also the benefit of a clean, easily interpreted voltage signal as discussed further herein below.

A second objective hereof is to provide a combined functionality to the user. While providing the necessary contact locations to the skin to achieve heart rate measurement, the apparatus can simultaneously serve as headphones for listening to audio from an audio source device such as a portable MP3 player, a radio, a mobile telephone (e.g., cellular, portable, satellite, etc.), etc. FIGS. 1, 2, and 3 illustrate this combined functionality in several arrangements.

To achieve this second objective, features and aspects hereof modify a typical headphone design. A portion of the headphone enclosure which is normally in contact with the skin in, on, or around the ear is made electrically conductive. This conductive headphone portion can serve as one of the required electrodes for voltage measurement. This headphone apparatus also serves for holding the electrically conductive surfaces, or electrodes, in place. The cables connecting the headphones to the audio source device also may serve to connect the electrodes to the heart-beat detection circuitry.

One exemplary embodiment is a modified in-the-ear style of headphones commonly known as ‘ear-buds’. Ear-buds are commonly worn one bud in each ear, such that the outer surface of each bud enclosure is in contact with the skin of the folds of the ear. In this embodiment, the outer surface of the bud enclosure is modified to be electrically conductive and made to serve as an electrode connected to the heart-beat detection circuitry. Some ear-bud designs, which are popular among exercisers, also contain a structure designed to fit around the ear, thus holding the ear-bud in place during vigorous physical activity. Such a design may also, in this embodiment, provide contact surfaces around the ear which may be used to hold a conductive surface (electrode) in constant contact with the skin around the ear.

Another exemplary embodiment hereof integrates the heart rate measurement function with some typical methods of carrying a portable audio source device. Exercisers who wish to wear a portable audio source device (MP3 player, radio, mobile telephone, etc.) frequently wear the audio source device in one of several locations: strapped to the upper arm, strapped to the wrist or forearm, clipped to the waistband of exercise clothing, held in the hand, etc. Features and aspects hereof may include an apparatus which holds the portable audio source device and the heart rate detection circuit in one of those convenient locations and integrates a conductive surface at that location to serve as one of the required electrodes connected to the heart rate detection circuit.

Several variations of the above exemplary embodiment are possible. The enclosure for the heart rate detection circuitry can serve as a holder for the portable audio source device, and can also be designed such that the back of the enclosure doubles as the contact surface, or electrode. Alternatively, said enclosure may be designed to clip onto an arm-band or waist-band in proximity to the audio source device, such that the clip, or the enclosure body itself, is held in contact with the skin and made to serve as an electrode. The enclosure may include an electrically conductive clip which, while holding the enclosure in place, also makes contact with conductive fabric built into the armband, and thus incorporating the armband's inner surface as the area in contact with the skin of the wearer.

The discussion below provides a number of exemplary embodiments, including combining the placement of required electrodes with a number of other common accessories or personal articles worn on the head. These articles include, for example: eyeglasses, sunglasses, goggles, hat, helmet or headband (e.g., any headwear apparel), a glasses lanyard or a goggles strap, etc. Any personal items that fit snugly on the head or in or onto the ear are candidates for a combined use of holding a required heart rate detection electrode in place.

The discussion below also presents a number of additional combined functionality benefits, including integrating elements needed for heart rate measurement with the following: equipment to allow the use of a mobile telephone, equipment required or recommended for an occupation or activity such as a helmet, equipment desired to be worn such as glasses, goggles or eyeglass lanyards. Combining heart rate measurement elements into products that are normally in contact with the skin of the head provides a benefit to the user by eliminating the need to wear an additional device on their person, such as a chest strap, solely for heart rate measurement.

In the figures to be described, a number of elements are common to several figures. These common elements are identified in Table 1 below with their associated reference numbers:

| Reference | |
|---|---|
| Number | Description |
| 1 | electrode (electrically conductive surface to be in contact |
| | with skin) |
| 2 | headphone |
| 3 | electrode (electrically conductive surface in contact with |
| | arm, torso or leg) |
| 4 | heart rate detection module |
| 5 | connection between heart rate detection module and audio |
| | source device |
| 6 | audio source device |
| 7 | audio ground wire |
| 8 | audio left channel signal wire |
| 9 | audio right channel signal wire |
| 10 | wireless transmission of heart rate data |
| 11 | wireless reception of heart rate data |
| 12 | receiver module |
| 13 | headphone speaker |
| 14 | DC Power line |
| 15 | wire connecting electrodes to heart beat detection circuitry |
| 16 | strap |
| 17 | waist area of wearer |

FIG. 1 illustrates an exemplary embodiment of a system wherein ear-bud style headphones 2 provide electrodes 1 located in one or both ears and an audio source device 6 is strapped to the arm of the wearer by an apparatus which provides another electrode 3 located on the arm. The electrodes 1 integrated with the headphones 2 are coupled by connection 15 to heart rate detection module 4 adjacent or integrated with audio source device 6. Connection 15 may be a wired connection or may be a wireless connection between the electrodes 1 and the heart rate detection module 4. Connection 15 may also provide connection of headphones 2 to audio source device 6 or a separate wired or wireless connection may be provided therebetween. This arrangement provides a benefit to the user by requiring no separate equipment attached to or in contact with his or her body to obtain a heart rate measurement, beyond that required for listening to audio via either stereo headphones or a single headphone in one ear. In other words, the familiar chest strap is no longer needed.

The heart rate detection module 4 contains circuitry for the calculation of heart rate and/or other physical parameters derived therefrom, such as respiratory rate from the timing between the detected heart beats signals on the electrodes 1 (and optionally 3). Another component of the system (not shown) may provide circuitry and logic for reporting or presenting the measured heart rate to the user or for applying the measured rate to a separate data storage apparatus (not shown) as described further herein below.

One aspect hereof provides for combining the required functions and elements above with existing devices, including, for example: audio headphones, eyeglasses and sunglasses, hats, helmets, headbands, goggles, goggle straps, eyeglass or sunglass lanyards, ear muffs, and other similar products which, in the normal usage, provide a close fitting to the head of the wearer. By virtue of this combination of functions, especially the positioning of an electrode or electrodes on the head through the normal usage of those products, the wearer is provided with a means of measuring heart rate and other derived data without the necessity of wearing any additional apparatus on their person.

FIG. 1 also shows an optional electrode 3 positioned on the user's arm and a presentation device 12 adapted to receive the measured information from the heart rate detection module for presentation to the user. The optional electrode 3 may also be coupled to the heart rate detection module 4 by connection 15 or may be integral with module 4. These additional features are discussed further herein below with reference to other exemplary embodiments. Before presenting other exemplary embodiments, some detailed aspects hereof are common to several of the embodiments. Some of these common aspects are discussed below.

### Electrodes

In many of the example embodiments that follow, one novel aspect arises in placement of electrically conductive surfaces, herein called ‘electrodes’. A clear definition of this element serves as a good starting point.

In accordance with features and aspects hereof an electrode (e.g., 1 or 3 of FIG. 1) has the following characteristics: it is comprised of an electrically conductive surface, designed such that it can be held in contact with the skin of the wearer, and with an electrical connection to the heart beat detection circuit 4. Examples of electrode material include any substance with good electrical conductivity, including metals such as copper, steel or aluminum, and also including other electrically conductive manufactured materials, such as electrically conductive rubber, plastics or fabrics. Nothing more is required of the electrodes except that they make good contact with the skin. Therefore, further details of their design can be determined by the specific embodiment as a matter of design choice.

The purpose of an electrode is identical to that of an ECG electrode as used in standard ECG technology, which is simply to provide contact to the skin at a certain location such that an electrical voltage potential can be measured between two or more locations on the body. While an ECG electrode could perform the functions of an electrode herein, ECG electrodes have some characteristics that are not required or desired for this application. ECG electrodes are universally made of an identical material, Silver/Silver Chloride, to insure that their conductivity is consistent across the industry. Such precision is not required in application of the features hereof. ECG electrodes are manufactured with an electrically conductive adhesive on one side, and are adhered to the skin at the desired location for the duration of the testing. In practice, the skin is prepared before an ECG electrode is affixed to it, again to insure a standard conductivity in repeated tests. For the purpose of heart rate monitoring as described herein, electrodes in accordance with this application can be made of any convenient material with a high coefficient of electrical conductivity, and they are not required to be glued in place. The skin is not required to be prepared before the electrode is positioned. A result of these more relaxed requirements is a greater variance in the signal characteristics, such as amplitude and noise, compared to an ECG signal. A clean, standardized waveform suitable for medical diagnostics would require adherence to industry standards in manufacture and placement of the electrodes. To accurately detect a heart-beat however, a simple electrode as described herein is sufficient. As used herein the term ‘ECG electrode’ refers to the medical industry standardized electrode, and the term ‘electrode’ refers to the simpler electrically conductive surface utilized in this application.

### Positioning of Electrodes

One exemplary arrangement of the above described electrodes is to have two electrodes positioned to be in contact with the skin of the head. Changes in the electrical voltage potential between or across the two electrode locations are produced during the normal functioning of the heart. The electrodes are connected by an electrical wire to heart beat detection circuit 4 which detects individual heart-beat signals therefrom. We can anticipate a nomenclature to be described in a later section, and label this arrangement of electrodes to be a lead of type H-H, meaning that the voltage measurement is taken between a position on the head (H) and another position on the head (thus H-H). The specific signal that is useful in identifying heart beats, termed the QRS complex, becomes more well-defined as the separation between the electrodes, increases.

An optional component of the system, the presence of which provides a greatly improved signal, comprises an electrode 3 positioned such that it is held in contact with the skin of the wearer on an arm, leg or a location on the torso. This electrode is either connected by a wire to the heart rate detection module 4, or else is integrated into the housing of it. If present, this electrode 3 is used in conjunction with the other electrode or electrodes 1 positioned on the head of the wearer, and together they provide the input to the heart rate detection module 4. While it will be shown that heart rate detection can be accomplished with electrode locations exclusively on the head (H-H), inclusion of an electrode at a location below the head can improve the detected signal significantly. In a later section, this arrangement will be termed a lead of general type H-AT, meaning that at least one electrode is located on the head, and another is located on the arm, leg or torso.

### Other Exemplary Embodiments

Placement of the electrodes can vary in accordance with various embodiments hereof. Suitable locations for an electrode positioned on the head of the wearer include the following locations: in the ear, on the ear, in contact with the skin of the head above, below or beside the ear, on the temple, on the side of the forehead, on the side of the back of the head. Contact with the skin is important; substantial interposition of hair between the skin and the electrode may produce less reliable results. Thus positions on the head with interposing hair would not be preferred due to diminished signal quality from the electrodes 1.

In terms of suitable locations for an electrode 3 positioned below the head of the wearer, the following exemplary locations may be used to produce a measurable reading of the voltage changes: on the shoulder, upper arm, lower arm, wrist or hand, on the torso, at the waist, on the chest or back, or on the leg. While all of these locations can produce a measurable signal, practical considerations for a sports and fitness or biofeedback tool integrated with an audio source device would suggest the bicep, wrist, hand or waist as preferred choices, these being locations where portable audio source devices are typically worn or carried, thus allowing for the incorporation of an electrode into the design of the audio source device body or a holder of the audio source device body, or held in place by the same armband, waistband, wristband or similar apparatus.

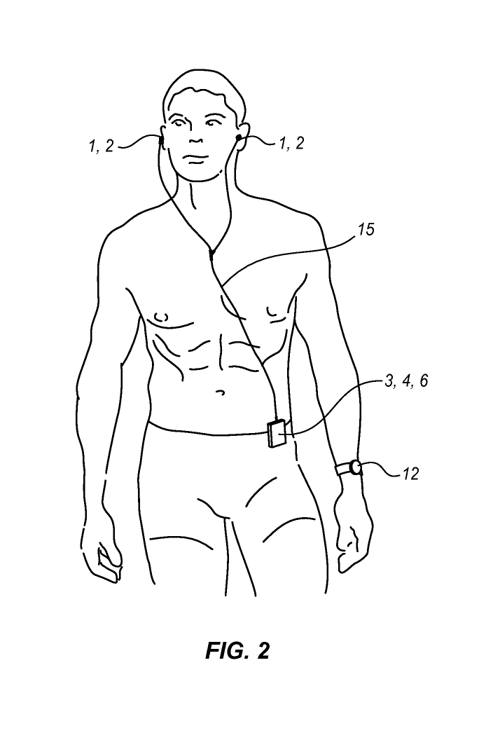

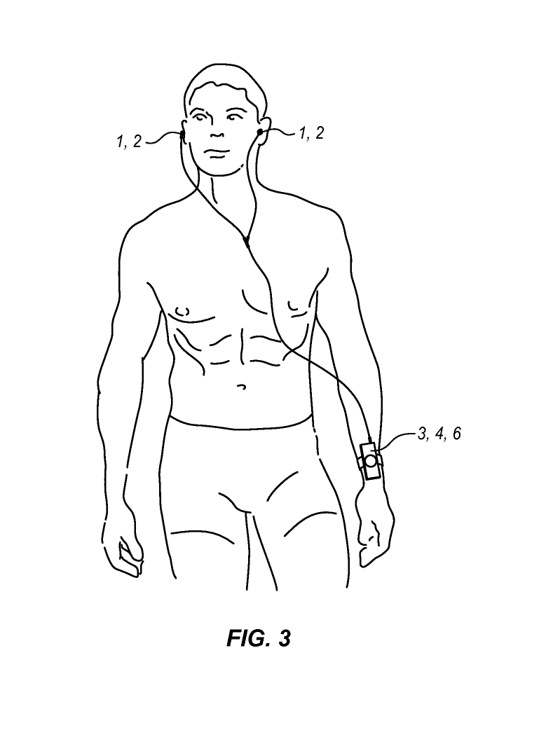

FIGS. 1, 2 and 3 show exemplary embodiments hereof in which an electrode 3 is integrated into the housing of the heart rate detection module 4, and that module 4 is designed such that it is held in place by an apparatus which also functions to hold a portable audio source device 6 in place. FIG. 1 shows an arrangement holding the module 4, the audio source device 6, and the optional electrode 3 in place on the upper arm. FIG. 2 shows an arrangement holding these items in place at the waist area 17 of the wearer. FIG. 3 shows an arrangement holding them in place at the wrist.

If the audio source device is strapped to the upper arm as in FIG. 1, the optional electrode 3 is designed such that it is held in place against the skin of the upper arm. If the audio source device is strapped to the wrist or forearm, the electrode 3 is designed such that it is held in place against the skin of the wrist or forearm. If the audio source device is clipped to the waistband of the clothing, the electrode 3 is held in place against the wearer's waist 17. This optional third electrode 3, whether in contact with the skin of the wearer at the upper arm, the wrist or forearm or hand, or the waist, either directly or through a moistened shirt, can be used in conjunction with the other electrode or electrodes 1 located on the head of the wearer. It should be noted that, when one electrode is located on the arm or torso in this fashion and another is located on the head, the periodic change in voltage from heartbeats is stronger and clearer (and thus more easily detected) than in embodiments where all of the electrodes are located on the head. In this embodiment, the signals can thus be analyzed with simpler circuitry or simpler algorithmic analysis, or can be analyzed with better accuracy, compared to a system with all of the electrodes located on the head.

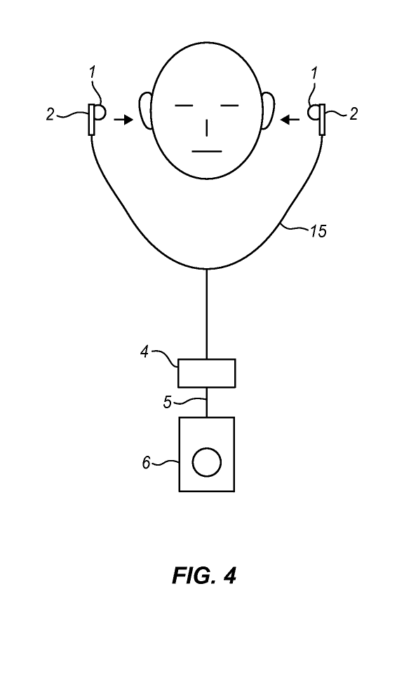

FIG. 4 shows a further detail of the system in which a connection 5 is made between the detection module 4 and the audio source device 6. This connection may be made by wires or a connector, or in some embodiments it may be made wirelessly. Power for the module 4 may be derived from the audio source device 6. A single cable may serve both to connect electrode(s) 1 to detection module 4, and also to provide wires 5 needed for audio production.

The detected heart beats are passed within the module 4 to an electronic circuit designed for analyzing the timing between individual beats, and deriving heart rate therefrom. The heart rate detection module 4 may also contain connections and/or circuitry designed to allow audio signals from an audio source device 6 to pass through to the headphones 2, either unmodified or modified by the addition of audio that communicates heart rate information to the wearer.

Another exemplary embodiment would affix or build in a conductive surface or surfaces to an on-the-ear style of headphones, or to an over-the-ear style of headphones. Said headphones may be of a style which holds the speakers in place via an apparatus which loops over the top of the ear and around the back of the head, or over the top of the head. Either a portion of one headphone, or a portion of each headphone, which is held in place against the skin of the ear during normal usage is modified to be an electrically conductive surface and made to serve as an electrode. Either an electrode on one ear with another electrode on the arm or torso, or a electrode on each ear, along with possibly another sensor on the arm or torso, make up the set of exemplary electrodes connected by electrical wires to the heart rate detection circuitry.

People who wish to exercise while listening to music, or telephone or other audio, and who wish simultaneously to know their heart rate while exercising, will benefit because they will no longer be required to wear a chest-strap to obtain their heart rate. Likewise, people who engage in relaxation, meditative or other therapies that involve music or other audio and that also involve heart-rate biofeedback will benefit from the improved comfort obtained from not wearing a chest strap.

Another objective hereof is to provide the user with a combined functionality, offering heart rate measurement combined with both music and telephone audio. A headset or headphones as described herein above may be connected to a device capable of serving as a mobile telephone and also capable of playing audio files or streamed audio, thus providing music or other audio in combination with telephone audio reception. Features and aspects hereof add heart rate monitoring to this combination by making a portion of the headset enclosure electrically conductive such as to serve as the required electrodes for heart rate measurement while not interfering with the audio production capabilities.

Another objective hereof is to provide the user with the heart rate measurement functionality by building the electrodes into an apparatus or article of clothing that he might wish to wear anyway, thereby making the measurement process less bothersome. One exemplary embodiment that serves this objective is to affix the electrodes to the inside of a hat, one sensor on either side, such that the electrodes would be held in place against the skin of the forehead, temple, above the ears, or at the back of the head. By virtue of the snug fit required to keep the hat in place, the electrodes are also held in place next to the skin. The heart rate detection circuitry can be built into the hat as well.

Another exemplary embodiment of a similar nature is that the electrodes may be affixed or built into the inner material of a headband, making contact with suitable locations on the head such as those described for a hat. The heart rate detection circuitry can be built into the headband.

In another exemplary embodiment, the electrodes are affixed or built into the inner surfaces of the ear-pieces of a pair of glasses or sunglasses such that the portion of the earpiece held in place in contact with the skin above or behind the ears serve as electrodes. The heart rate detection circuitry can be built into the frame of the glasses.

In another exemplary embodiment, the electrodes are affixed or built into the ends of a lanyard intended for keeping glasses or sunglasses around the neck of the wearer when not worn on the face. The design of some of these products is such that one end of the lanyard slips over and envelopes each earpiece of the glasses. When the glasses are being worn on the face, the inner surfaces of the lanyard ends are held firmly in place against the skin of the wearer above or behind the ears. The portion of each lanyard end which is in contact with the skin can be an electrode. The heart rate detection circuitry can be built into the lanyard.

In another exemplary embodiment, the electrodes are affixed or built into the inner surfaces of a set of ear-muffs which surfaces come into contact and are held in place against the skin of the ear during normal usage. The heart rate detection circuitry can be built into the ear-muffs.

People who wish to monitor their heart rate and who may normally wish to wear any of the above items of clothing, hats, eyeglasses, sunglasses or glasses lanyards, or any other article that provides a snug fit to the head will benefit by not needing to wear a chest strap for heart rate measurement.

Another objective hereof is to provide the user with the heart rate measurement functionality by building the electrodes into an apparatus or article of clothing that he might need to wear anyway in the course of his employment or activity, thereby making the measurement process more rigorously enforced. One exemplary embodiment that serves this objective is to affix the electrodes to the inside of a helmet such as is worn for football, or in the military, or during dangerous activities such as rock climbing, bicycling, or motorcycling, or a safety helmet at construction sites. The electrodes, at least one electrode on each side, are affixed or built into the helmet such that the electrodes would be held in place against the skin of the forehead, temple, above the ears, or at the back of the head. By virtue of the snug fit required to keep the helmet in place, the electrodes are also held in place next to the skin. The heart rate detection circuitry can be built into the helmet as well, in another location. For purposes of monitoring by external personnel, the circuitry necessary to transmit the heart rate information to a distant receiver can be built into the helmet. In this way, the coaching staff along the sidelines, for example, or the health officers at a military command post, can monitor the heart rate and associated data of the wearer without requiring the wearer to wear any additional equipment.

Another objective hereof is to provide the wearer with heart rate measurement functionality during activities that require the wearing of a mask or set of goggles. Such activities include, for example, swimming, scuba, and skiing. During normal usage, the mask or goggles are held fast to the face, typically by an elasticized strap positioned around the head. An exemplary embodiment would affix or build in electrodes, at least one on each side, into the sides of the mask, or into the strap, such that they would make contact with the skin of the head beside, above or below the eyes, or at the temples, or above or behind the ears. The heart rate detection circuitry can be built into the mask or goggles as well. Persons wishing to measure their heart rate while engaged in activities for which they would normally wear a mask or goggles anyway will benefit by not needed to wear an additional apparatus such as a chest strap in order to obtain heart rate information.

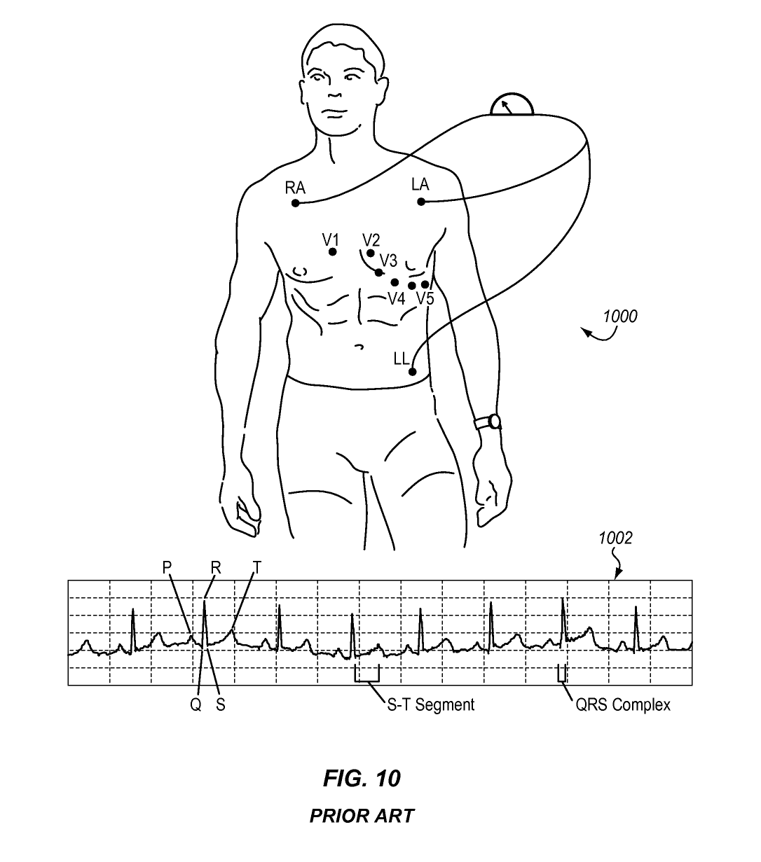

Another benefit hereof is to provide the user with additional data derived from precise heart beat measurement. In current practice, if the timing between individual beats is sufficiently precise, additional physiological data can be obtained from it. This style of analysis is possible only if the precise timing of a signature element of an ECG signal is obtainable, which is the case with this apparatus. This explanation borrows from standard ECG terminology discussed herein below. In most ECG views of the heart, one signal spike is predominant, as by far the highest point. In standard ECG terminology it is called the ‘R’ point, or simply ‘R’, part of a group of named points in the signal called the ‘QRS Complex’ as generally known and as shown in FIG. 10. The R point is unique within the ECG signal: because of the sharp rising edge and sharp falling edge, ‘R’ provides a sharply defined moment in time, which can be captured by an electronic circuit and thus can be precisely timed. Various other features of the ECG signal show more rounded shapes and do not permit the same precise timing. Likewise, other technologies as described herein above, which do not make use of the electrical signals of the heart, are found not to have such a precise measuring point. The number of milliseconds between one ‘R’ point and the next is variously referred to as ‘R-R variability’ or ‘beat-to-beat variability’, and it has become the basis for various types of analysis sometimes called ‘beat-to-beat analysis’. It is only possible however, in an apparatus such as the one herein described that measures the electrical signals of the heart in a manner to make the QRS complex generally, and the R point specifically, available for capture and timing. One example of this style of analysis is respiratory rate. This data is derived from the heart beat signal and the timing of individual heart beats, as determined by the heart rate detection circuitry. During a period of time when the subject's heart rate would be considered constant, for example, a slight increase in heart rate is detectable during inspiration, and a slight relative decrease in heart rate is found during expiration. The apparatus described herein is measuring a voltage signal generated by the heart, and, as will be shown later, the signal obtained therefrom, although perhaps different from normal ECG views, still shows a predominant QRS complex and R point. This allows for the precise timing of individual heart beats as required to detect slight variations. Thus, the respiratory rate, or breathing rate of the wearer can be calculated and presented to the user along with heart rate. In like manner, other physiological data derived from R-R variability or beat-to-beat variability is also capable of being obtained from this apparatus.

People who engage in exercise and who wish to know their respiratory rate or other physiological data derived from beat-to-beat timing will benefit from this feature. Also, people who engage in relaxation, meditative or other therapies and who wish to know their respiratory rate as biofeedback data will benefit. Additionally, people who wish to know other physiological data that are derived from beat-to-beat analysis will benefit.

If the wearer desires to carry the heart rate detection module 4 clipped to the waistband, and has a shirt between the waistband and the skin, contact 3 can be designed such that it is held in place against the shirt material at the waist, and the shirt material can be moistened to allow the electrical signal to pass through it. Another design option to accommodate wearers who clip the audio source device to the waistband with a shirt between waistband and skin, is to design the torso contact such that it is held against the skin at the waist, by the tension of the clothing waistband, and connected by a wire to the heart rate detection circuitry clipped to the waistband, which wire passes through the fabric of the shirt. Yet another design option to accommodate wearers who clip the audio source device to the waistband with a shirt between waistband and skin, is to design a conductive area into the fabric of the shirt at an appropriate location such that the shirt fabric at that location is conductive from one side of the shirt material to the other without requiring to be moistened. This conductive area on the shirt can be positioned such that, with the shirt tucked in, the conductive area is positioned at waist height, at such a position corresponding to the location of the waist clip holding the heart rate detection circuitry and possibly also the portable audio source device. The shirt then acts as a pass-through conductor, making a connection between the skin of the wearer and the electrode built into the enclosure of the heart rate detection module.

Likewise, if the wearer desires to carry the heart rate detection module 4 on the arm on the outside of a sleeve or as part of a sleeve, similar solutions as the above can provide the connection needed through the fabric of the sleeve. Another possible embodiment for the sleeve is to build a pocket for an audio source device into the sleeve in a manner that holds the fabric of the sleeve snugly in contact with the skin of the arm, and, by means of conductive fabric in the sleeve at that point, or some other method, provides for placement of electrode 3 on the arm. This solution is suitable for colder conditions where the user may want access to the controls on the audio source device, but desires to wear long sleeves.

### Points and Leads

In ECG terminology, a ‘point’ is a carefully specified location on the body where an electrode is affixed, and a lead is a ‘view’ of the heart obtained by taking a voltage measurement across two or more points. The points in current use

Features and aspects hereof are expressed herein in terms of several new points of contact, and several new leads derived therefrom. Note that these points are less specific than traditional ECG points. This is allowable because the resulting waves as used herein are intended only for heart-beat detection and timing (and derived data such as respiratory rate determination), not to illustrate various pathologies to a cardiologist. Subtle changes in the waveform due to imprecise positioning of the contact point do not detract from the intended function. With this generalized positioning in mind, note especially the use of a ‘g’ prefix for the ‘generalized left arm’ (gLA) and the ‘generalized right arm’ (gRA). This indicates that any location on the arm or hand is sufficient, and avoids confusion with the standard named ECG leads ‘LA’ and ‘RA’ which have precise positioning requirements. All of the other new points defined here, such as ‘left head’ (LH) are also generalized in that their position does not need to be precisely specified in the same manner that leads for an ECG test are precisely positioned. As stated earlier, this is due to the limited intended usage of this apparatus: instead of a diagnostic waveform, we seek only an identifiable QRS complex and R point suitable for detecting and timing heart beats. The ‘g’ prefix, however, has only been added where needed to form a unique point name, different from existing terminology.

New points are described as follows:

Point LH (left head) is any point of contact with the left side of the head, including the skin in, on or around the left ear, or above or behind the left ear, or on the left temple or left forehead, or the back left side of the head.

Point RH (right head) is any point of contact with the right side of the head, including the skin in, on or around the right ear, or above or behind the right ear or on the right temple or right forehead, or the back right side of the head.

Point H (head) encompasses the definitions of LH and RH.

Point gLA (generalized left arm) is any point of contact on the left arm, from the shoulder down to the digits.

Point gRA (generalized right arm) is any point of contact on the right arm, from the shoulder down to the digits.

Point T (torso) is any point of contact on the leg or torso.

Point AT (arm or torso) is any point of contact on the arm, leg or torso, thus encompassing point definitions gLA, gRA and T.

### New Lead Definitions

With the new point definitions presented above, we can now define new leads. Any lead which is defined as a combination of the above defined points, and which involves one of the points on the head as a necessary component, fits within the unique claims hereof. These fall into two general categories: H-AT is a lead that involves a point or points of contact H (on the left head or right head) and another point or points of contact AT (on the arm, leg or torso). The second general category H-H is a lead that involves a point or points of contact H (on the head) and another point or points of contact H (on the head). Note that in both of these general cases, at least one point on the head is a preferred approach to providing the monitoring, and is one element of the uniqueness of the apparatus.

Under the generalized lead category H-AT, examples of possible new leads include:
gLA-H—point(s) on the left arm and point(s) on the head
gLA-LH—point(s) on the left arm and point(s) on the left head
gLA-RH—point(s) on the left arm and point(s) on the right head
gRA-H—point(s) on the right arm and point(s) on the head
gRA-LH—point(s) on the right arm and point(s) on the left head
gRA-RH—point(s) on the right arm and point(s) on the right head
T-H—point(s) on the leg or torso and point(s) on the head
T-LH—point(s) on the leg or torso and point(s) on the left head
T-RH—point(s) on the leg or torso and point(s) on the right head

Under the generalized lead category H-H, examples of possible new leads include:
LH-RH—point(s) on the left head and point(s) on the right head

### Point and Points

The definitions above define a very broad range of possibilities, especially with the inclusion of the term ‘point(s)’, indicating one or more points. Generally speaking, a voltage potential is thought of as a measurement across two points. In electrocardiography however, a more complex arrangement is commonly used: multiple points are connected together to form one side of the measurement. This is termed an ‘indifferent electrode’ as it shows a lesser influence from the actions of the heart than the ‘exploring electrode’ which is positioned over the region of interest. This arrangement of an indifferent electrode and an exploring electrode is used throughout the standard ECG leads in current electrocardiography. One traditional grouping of locations LL, LA, and RA connected together form what is called a ‘central terminal’ which is used as one side of the measurement in many standard leads. It acts as the optimal indifferent electrode because it shows almost no effect on its own, in many ways similar to a ground. For example, the standard limb lead VR as presently practiced measures between the central terminal on one side of the galvanometer and RA (right arm) on the other.

In the leads defined above, the left and right sides of the ‘-’ (dash mark) are to be interpreted as opposing inputs to the galvanometer, or voltmeter, used for measuring the voltage potential. Thus for example, in a T-H case, while multiple points may be positioned on the body to form the ‘T’ electrode, these are understood as being connected to form a single electrode. Likewise, the ‘H’ electrode, by the definitions above, may involve multiple physical points on the head connected together which act as a single electrode. By the location of the dash mark (‘-’), the actual voltage measurement takes place between the ‘T’ electrode and the ‘H’ electrode. This demonstrates that the head locations are not superfluous, but rather are exemplary, preferred locations for the measurement. The incorporation of multiple physical points is a well-understood method for reducing common mode noise, and is used throughout current electrocardiography practice. As compared to prior techniques and devices for measurement such as ECG measurements from the torso, features and aspects hereof provide for at least one electrode at a contact point on the head.

An example of this, and a nomenclature describing it for the purposes hereof, can be shown as: (gLA,LH)-RH. Leads in this format can be read as: point(s) on the left arm and point(s) on the left head are combined to act as one electrode, and point(s) on the right head act as the other electrode. Note the use of parentheses to show a grouping, and the use of the dash (‘-’) separating the two inputs to the voltage measurement circuitry. With the above understanding in place, it is also possible to define additional useful leads derived from the new point locations, including but not limited to:
(gLA,LH)-RH—point(s) on the left arm and point(s) on the left head, measured against point(s) on the right head
(T,LH)-RH—point(s) on the torso and point(s) on the left head, measured against point(s) on the right head
(gRA,RH)-LH—point(s) on the right arm and point(s) on the right head, measured against point(s) on the left head
gLA-(LH,RH)—point(s) on the left arm measured against point(s) on the left head, and point(s) on the right head, where the two head conductors are combined to act as one electrode
T-(LH,RH))—point(s) on the torso measured against point(s) on the left head, and point(s) on the right head, where the two head conductors are combined to act as one electrode

### Demonstration of New Leads

The following specific examples of tracings obtained from a healthy subject during ECG examination, utilizing the new lead definitions above, are selected to demonstrate that, given the broad definitions of points used above, anyone with a normal skill in the art can obtain a usable signal suitable for heart rate measurement from these leads. In these examples, a standard ECG machine was used, and the output of ECG standard limb lead 2 (lead II) from the ECG machine is shown. This lead utilizes the ECG probes labeled LL (left leg) and RA (right arm) and LA (left arm). A standard ECG machine requires these three probes to be connected, using the third point (LA in lead II, for example) to reduce common mode noise, and will not operate if any of these probes are disconnected. In the following examples, probes LL, RA and LA on the testing machine were utilized to obtain a signal. Their positions however are not in general the normal ECG locations for these probes. In the following examples LL and LA are frequently connected together on the same electrode, to circumvent the “lead-off detection” built into the ECG machine. It should be clear that in examples showing two probes attached to the same electrode, and another probe attached to a different electrode, only two electrodes are required for a successful embodiment.

FIGS. 10 through 18 show the output from a standard ECG machine with probes connected to a human subject in various configurations. The filters and other signal processing required for standard ECG measurement are well known to those of ordinary skill in the ECG art, and thus provide a baseline of information to which features and aspects hereof add. In some cases, the standard ECG circuit may provide all of the essential filtering and other signal processing required for detecting a heart rate in the exemplary embodiments. While a standard ECG machine is not directly useful for a device applying features and aspects hereof, the ECG machine is a useful tool for providing clear demonstration of the various lead configurations proposed by features and aspects hereof. Unless indicated, the tracings were recorded at settings of 10 mm/mV (10 millimeters on the graph in the Y direction represent 1 millivolt of signal amplitude) and 25 mm/s (25 millimeters on the graph in the X direction represent 1 second of time).

FIG. 10 shows a probe configuration 1000 and resultant tracing 1002 as measured by a standard ECG machine as presently practiced with three probes (LL (left leg), LA (left arm) and RA (right arm)) in place in the approximate traditional locations for standard ECG measurement for cardiology purposes, and showing the output known in cardiology as lead II. Also in FIG. 10, the standard V lead points are shown on the subject's torso, from V1 through V6. These make up the standard electrode points for standard ECG measurement. Also in FIG. 10, illustrated on the tracing, standard components P, Q, R, S and T of the cardiac waveform are labeled, as are the QRS complex and the ST segment, as they are commonly known. Usage of these demarcations is standardized in the cardiology discipline and well known to those of ordinary skill in the art. They are presented herein merely as a background for definitions of new points in accordance with features and aspects hereof.

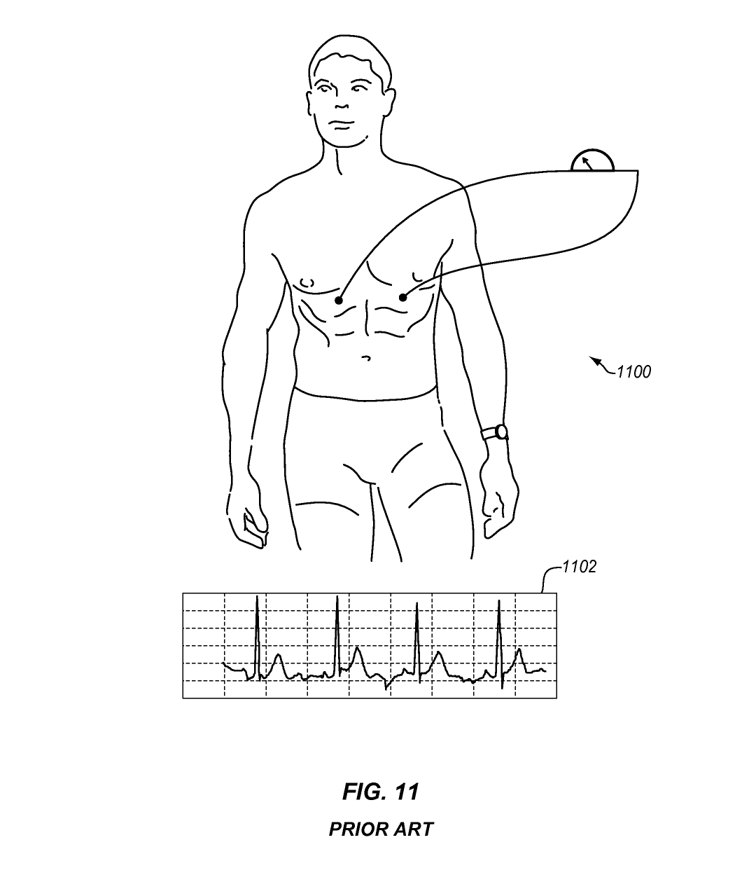

FIG. 11 shows another probe configuration 1100 and resultant tracing 1102 as measured by a standard ECG machine as presently practiced with three probes (LL (left leg), LA (left arm) and RA (right arm)) placed in the approximate traditional locations for a chest strap method of heart monitoring. Probes LL and LA were positioned at the left chest where one electrode of the chest strap rests, connected together, and probe RA was positioned at the right chest, where the other side of a chest strap rests. This configuration is common and shows the type of signal that a chest-strap based heart monitor would encounter and use. This tracing was recorded as 20 mm/mV, double the normal amplification.

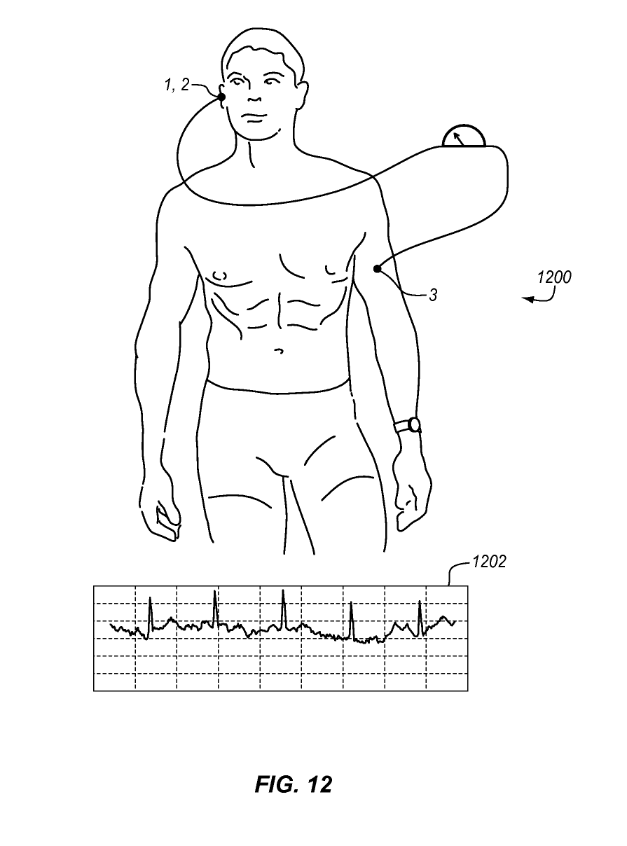

FIG. 12 shows a probe configuration 1200 and resultant tracing 1202 as measured by a standard ECG machine, with two conductors 1 and 3 as described in one of the exemplary embodiments above. In this case probes LL and LA are connected together and attached to a conductor on the left bicep, and probe RA is attached to a conductor in the right ear, such as one side of an ear-bud headphone. Some detailed aspects of the cardiac waveform shown above with respect to FIGS. 10 and 11 are not visible in this example. The QRS complex however, the high rising element, is prevalent. The QRS complex is the only signal required for detection of heart beats and determination of heart rate, and also the only signal required for precise R-to-R (beat-to-beat) timing as needed for respiratory rate calculation and other derived physiological data. This is an example of a new lead of the form H-AT, with a point or points on the head, and another point on the arm or torso. Specifically, this is newly defined lead gLA-RH.

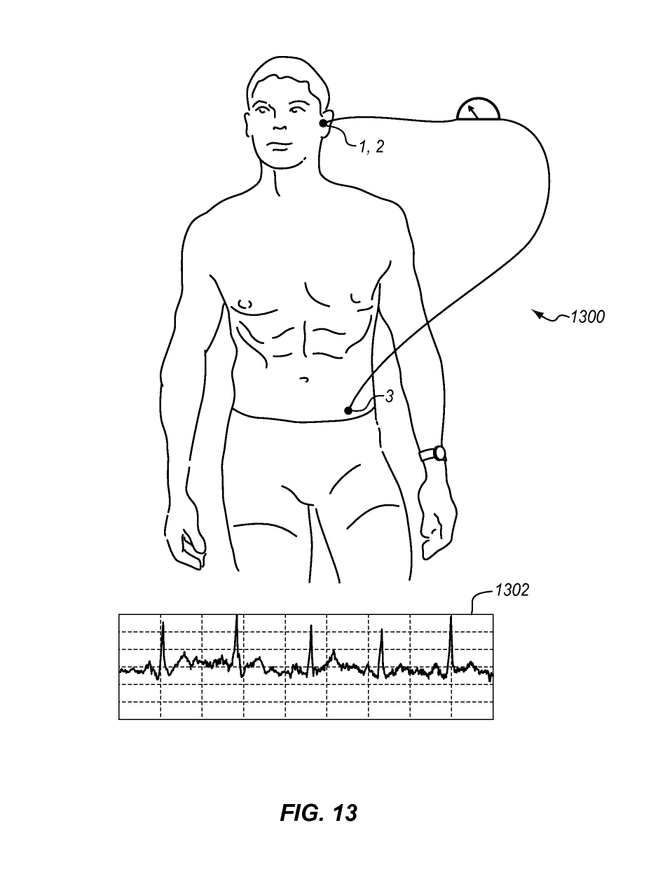

FIG. 13 shows a probe configuration 1300 and resultant tracing 1302 as measured by a standard ECG machine, with two conductors 1 and 3 as described in one of the exemplary embodiments above. In this case probe LL and LA are connected together and attached to a conductor on the left waist, and probe RA is attached to a conductor in the left ear, such as one side of an ear-bud headphone. This is an example of a new lead of the form H-AT, with a point or points on the head, and another point on the arm or torso. Specifically, this is newly defined lead T-LH. This tracing was recorded at 20 mm/mV.

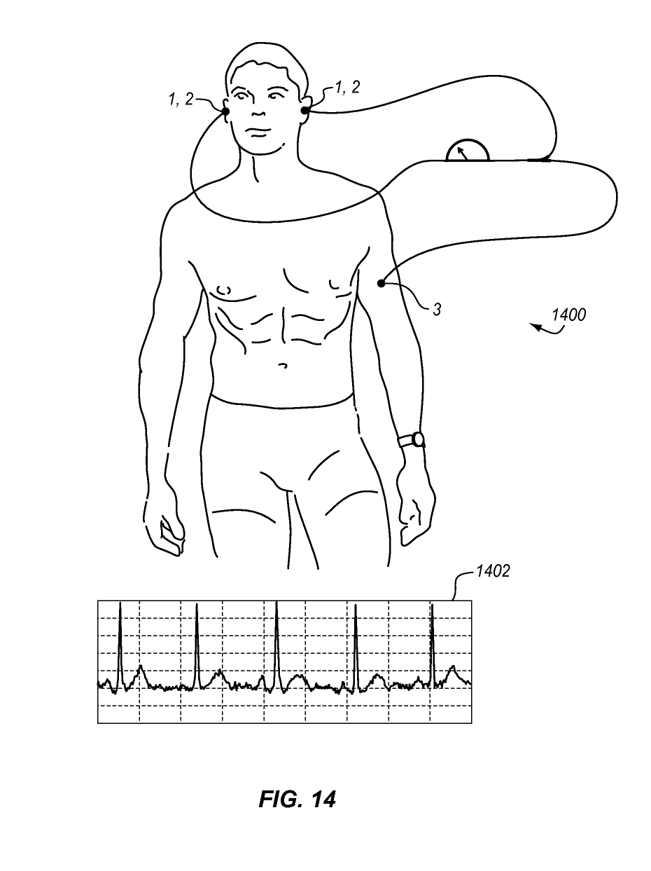

FIG. 14 shows a probe configuration 1400 and resultant tracing 1402 as measured by a standard ECG machine, with three conductors (1, 1, and 3) as described in one of the exemplary embodiments above. In this case, probe LA was attached to a conductor in the left ear, and probe RA was attached to a conductor in the right ear, such as when built into a pair of ear-bud headphones. The third probe (LL) was attached to a conductor in contact with the left bicep of the wearer. This is another example of the generalized format H-AT. More specifically, this is lead would be defined as (gLA,LH)-RH where the left arm and left ear are combined to act as a single electrode. This tracing was recorded at 20 mm/mV.

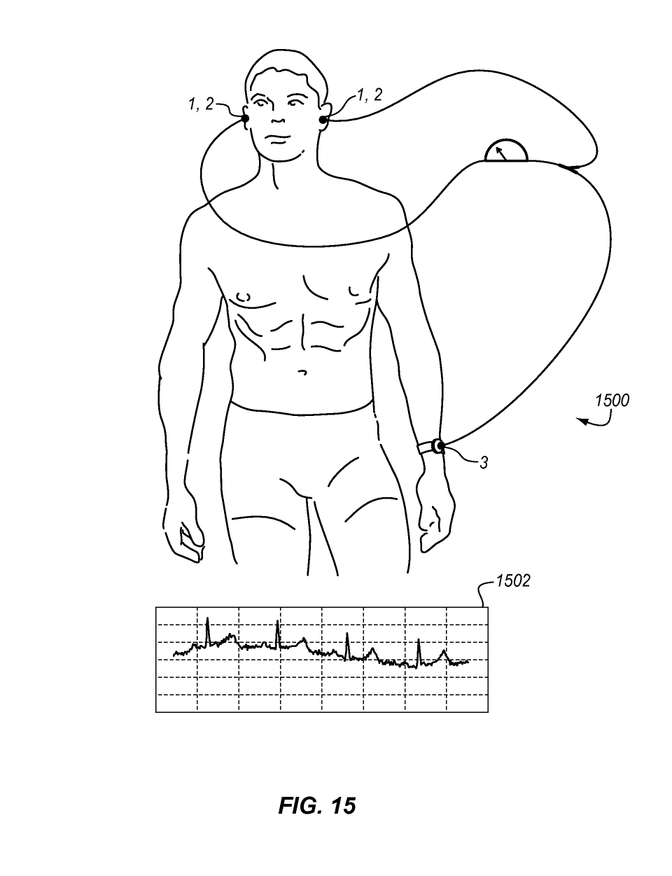

FIG. 15 is a probe configuration 1500 similar to FIG. 14 except that the third probe (LL) was connected to a conductor 3 in contact with the skin of the wrist of the wearer. This is the same new lead definition as in FIG. 14 (gLA,LH)-RH, taking advantage of the generalized definition of gLA to move the conductor to the wrist. It is included here to demonstrate that the location on the arm of the gLA point does not significantly alter the signal, for heart rate monitoring purposes. That is, the QRS complex is still prominent relative to the rest of the signal, and sufficient for detecting individual heart-beats and the timing between them. This is a variant of generalized lead H-AT. This tracing 1502 was recorded at 10 mm/mV, compared to 20 mm/mV in FIG. 14.

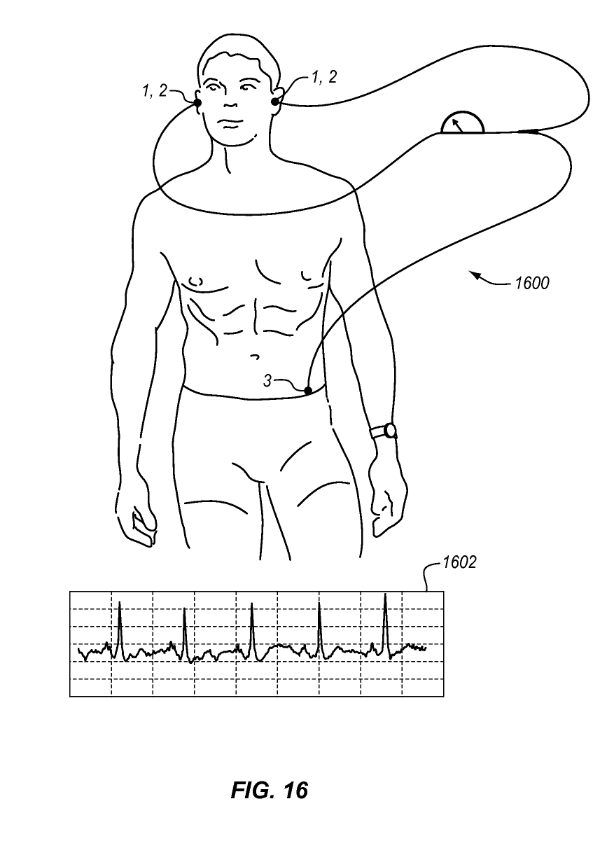

FIG. 16 is a probe configuration 1600 similar to FIG. 14 except that the third probe (LL) was connected to a conductor 3 in contact with the skin of the waist of the wearer, to the left side, instead of the arm. This is specifically defined as new lead (T,LH)-RH and is another example of the generalized new lead H-AT. It is included to demonstrate that the location of the AT point on the torso instead of the arm does not significantly alter the signal, for heart rate monitoring purposes. This tracing 1602 was recorded at 20 mm/mV.

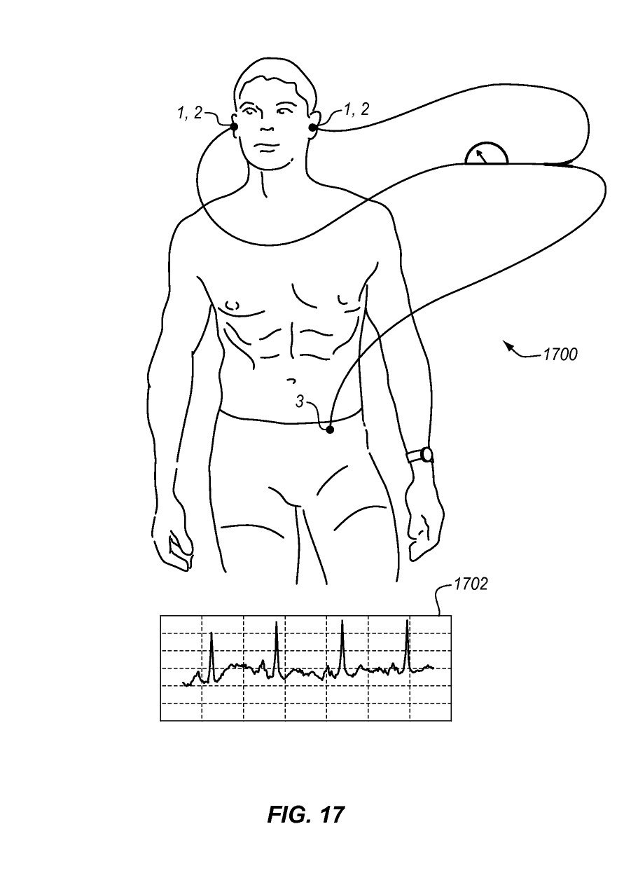

FIG. 17 is a probe configuration 1700 similar to FIG. 16 except that the LL probe was connected to a conductor held in place by the wearer's waistband, and the wearer's shirt (not shown) was tucked in such that it was between the conductor and the skin. The shirt was moistened with a small amount of water at the location of the conductor. This modified H-AT configuration is included to demonstrate the practical feasibility of monitoring heart rate in sports and fitness situation where clothing is involved, simply by dampening the fabric between skin and conductor. The resultant trace 1702 was produced.

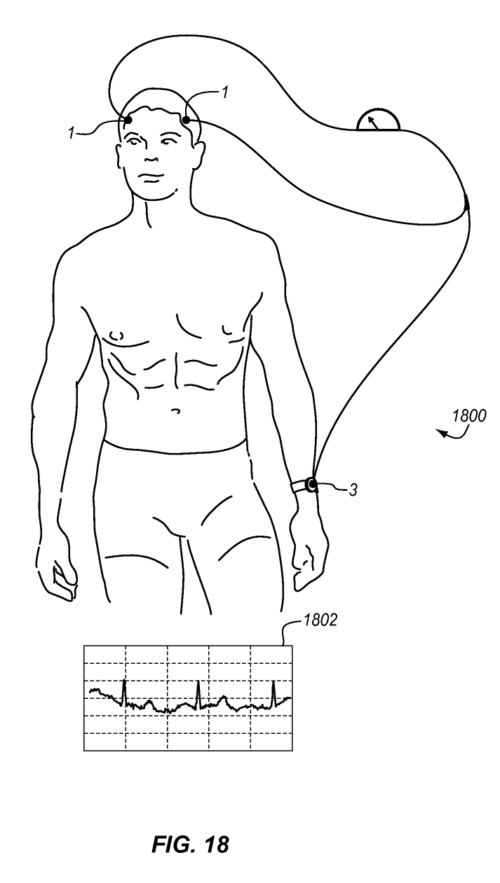

FIG. 18 shows a configuration 1800 and tracing as measured by a standard ECG machine, with three conductors as described in one of the exemplary embodiments. In this case, two conductors 1 and 1 were positioned one on each temple, as when built into a hat-band. The third sensor 3 was attached to the left wrist of the wearer. This demonstrates the degree of latitude that can be accepted in placement of the head points LH and RH, when compared to FIGS. 12-17. Placement of the head electrodes on the temple does not alter the signal significantly compared to a placement in the ears, for heart rate monitoring purposes. This is another H-AT configuration. This tracing 1702 was recorded at 10 mm/mV.

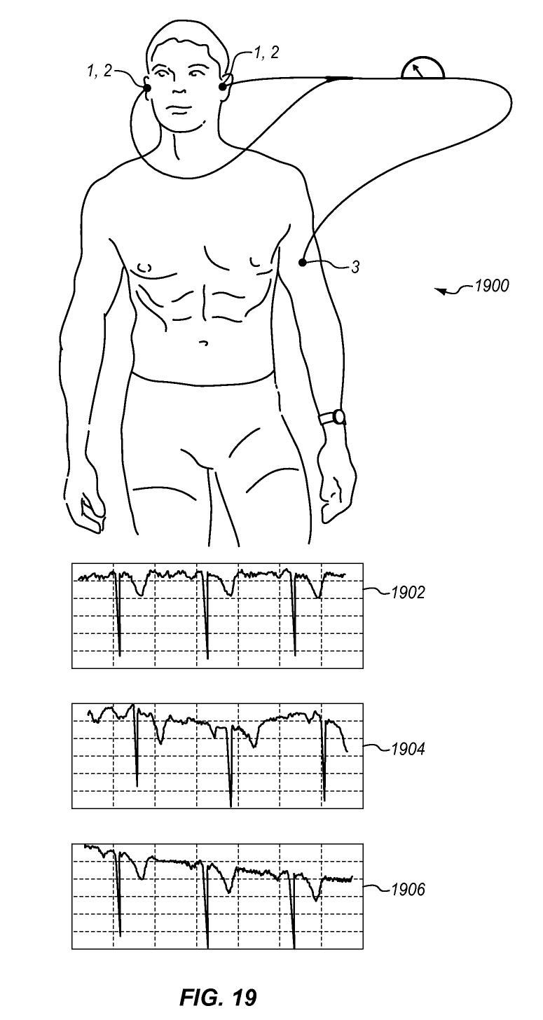

FIG. 19 shows a configuration 1900 and tracings 1902, 1904, and 1906 as measured by a standard ECG machine, with three conductors (1, 1, and 3) as described in one of the exemplary embodiments. In this case, probe LL is attached to a conductor positioned in the left ear, probe LA is attached to a conductor positioned in the right ear, and the two probes are connected together to act as a single electrode. Probe RA is attached to a conductor on the left bicep. This lead, of generalized type H-AT and specifically (LH,RH)-gLA provides a unique advantage over some other leads. With the left head and right head combined and acting as one electrode, and the arm acting as the other electrode, the signal is still obtained if the user removes one of the ear-buds to have a conversation, or if the user mistakenly places the right ear bud in the left ear and vice versa. Trace 1902 shows the tracing when both ear conductors are positioned in the ears. Trace 1904 shows the signal when the right ear conductor (probe LA) is removed during recording and trace 1906 shows the signal when the left ear conductor (probe LL) is removed during recording. Note that the polarity has reversed in this example compared to the other tracings. This is merely a function of having switched the ECG probes to the opposite sides of the body from the ECG manufacturer's intent. It has no impact on the usefulness of the signal. These were recorded at 20 mm/mV.

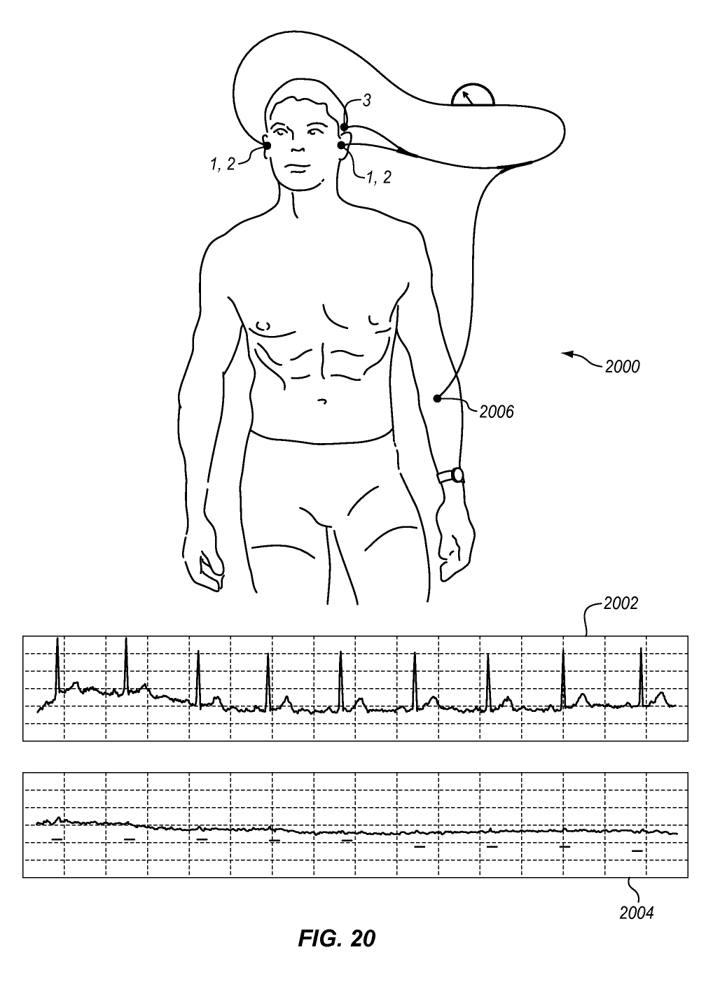

FIG. 20 shows a configuration and tracings as measured by a standard ECG machine, with four conductors in place. Three of the conductors (1, 1, and 3) are as described in one of the exemplary embodiments, and the fourth (2006) is present to provide a reference tracing. The two tracings shown in FIG. 20 (2002 and 2004) were captured simultaneously from the same human subject. Trace 2002 shows the output of the reference lead, with heart beat signals prevalent. Trace 2004 shows the output of one exemplary embodiment which does not make use of a conductor (2006) on the arm or torso. In this tracing (20b) two probes (LA and RA) were attached to conductors (1 and 1) placed in the ears, LA in the left ear, and RA in the right ear. The third probe (LL) was attached to a conductor 3 placed just behind the left ear. The lower tracing then is an example of the second general type of new lead: H-H, where all of the contact points are on the head. Specifically it is of type (LH,LH)-RH. It is perhaps not immediately clear that a signal is present in the lower tracing, but a careful comparison with the top tracing shows that, at each heart beat location (QRS complex), a slight change in amplitude is present. Although the amplitude of the signal is not much above the noise, a frequency signature is also present during a heart beat, which can be isolated with the proper filtering and other signal processing techniques. Standard techniques used for ECG measurement would not be sufficient to retrieve the heart beat signals from the lower tracing. Additional specialized filtering and signal processing is necessary as described below to isolate and identify the QRS complex in this high noise embodiment. This was recorded at 20 mm/mV.

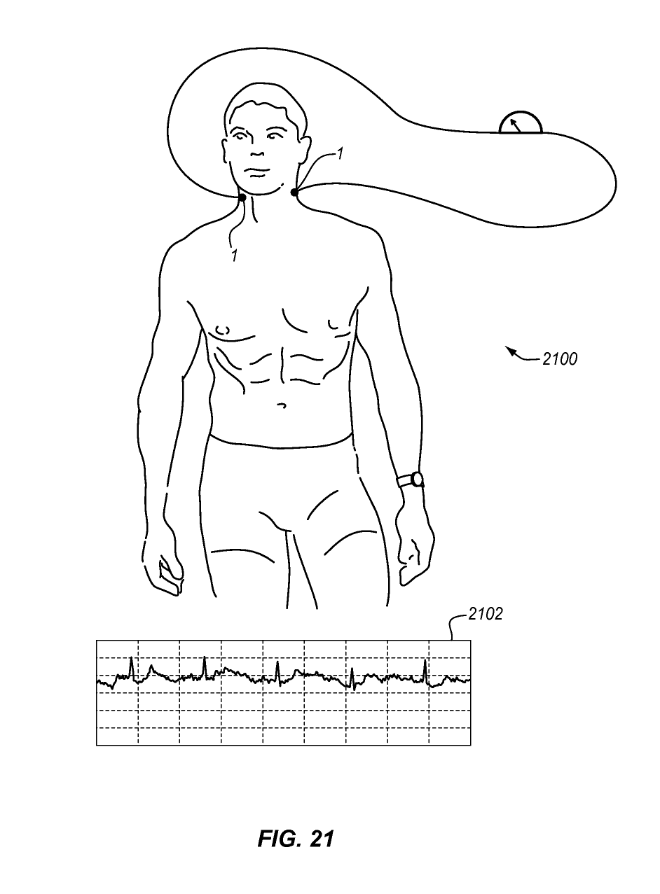

FIG. 21 shows a probe configuration 2100 and resultant tracing 2102 as measured by a standard ECG machine, with two conductors (1 and 1) as described in one of the exemplary embodiments. In this case, probes LL and LA are attached to the same conductor, positioned very low on the left side of the head (e.g., near the base of the skull or jaw bone), such that the two probes are connected together to act as a single electrode. Probe RA is attached to a conductor positioned on the right, again low on the side of the head. This lead is of generalized type H-H and specifically is lead LN-RN. This was recorded at 20 mm/mV.

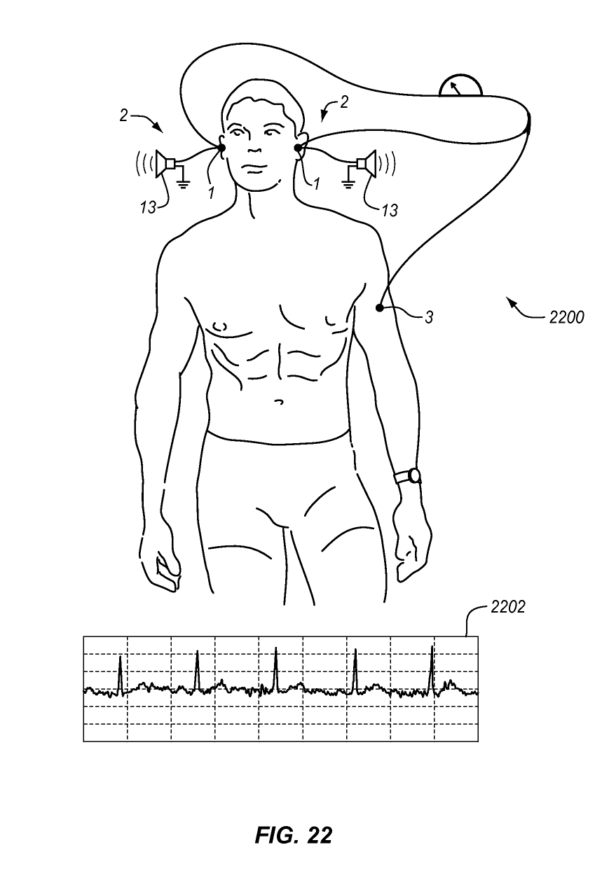

FIG. 22 repeats an earlier experiment (shown in FIG. 14) and shows a probe configuration 2200 and resultant tracing 2202 as measured by a standard ECG machine, with three conductors as described in one of the exemplary embodiments above. In this case, probe LA was attached to a conductor 1 in the left ear, and probe RA was attached to a conductor 1 in the right ear, such as when built into a pair of ear-bud headphones (2 and 13). The third probe (LL) was attached to a conductor 3 in contact with the left bicep of the wearer. This is example of the generalized format H-AT. More specifically, this is lead would be defined as (gLA,LH)-RH where the left arm and left ear are combined to act as a single electrode. This time, however, the LA probe was also connected to the left channel audio signal wire of the headphones, and the RA probe was also connected to the right channel audio signal. Music was played on the headphones while the tracing was made. This demonstrates that it may not be practical to utilize the signal wires used to play audio to connect the electrodes to the heart beat detection circuitry. This tracing was recorded at 10 mm/mV.

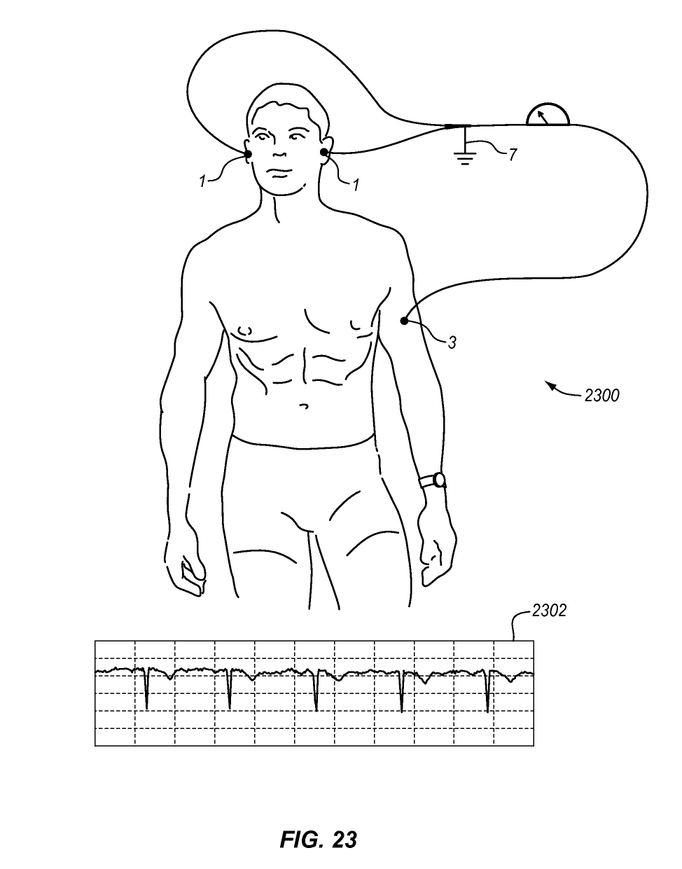

FIG. 23 repeats an earlier experiment (shown in FIG. 19) and shows a probe configuration 2300 and resultant tracing 2302 as measured by a standard ECG machine, with three conductors as described in one of the exemplary embodiments. In this case, probe LL is attached to a conductor 1 positioned in the left ear, probe LA is attached to a conductor 1 positioned in the right ear, and the two probes are connected together through a ground of the audio signals (7) to act as a single electrode. Probe RA is attached to a conductor 3 on the left bicep. This lead is of generalized type H-AT and specifically (LH,RH)-gLA. In this experiment, the ground wire for both left and right headphones was also connected to probes LA and LL, and music was played. This demonstrates that it may be practical to utilize the same ground wire that connects the audio headphones to connect the electrodes to the heart beat detection circuitry, depending on the requirements of that circuit. This tracing was recorded at 10 mm/mV.

### Filtering and Signal Processing Considerations

A comparison of the tracings in FIGS. 10 and 15 demonstrates a comparative difference between the standard ECG output and the output of one of the exemplary embodiments. FIG. 10 was recorded with probes LL (left leg), LA (left arm), and RA (right arm) positioned in the traditional locations for ECG measurement, and thus represent present practices. FIG. 15 was recorded with probe LL at the left wrist, LA in the left ear, and RA in the right ear, which is a generalized new lead case H-AT, and specifically describes lead (gLA,LH)-RH. Both were recorded at a setting of 10 mm/mV. The amplitude of the QRS complex shown in FIG. 10 is significantly higher than in FIG. 15. In FIG. 10 (traditional lead location) the QRS complex shows a rise of approximately 20 mm, corresponding to 2 mV. By comparison, FIG. 15 (leads positioned as per one of the exemplary embodiments hereof) shows QRS complex amplitude of 10 mm, corresponding to about 1 mV. Also, the relative amplitude of the QRS complex compared to the rise of the ST segment is greater in FIG. 10, showing nearly a 3 to 1 ratio, while the same comparison in FIG. 15 shows about a 1.5 to 1 ratio. These differences demonstrate the potential need for greater amplification, and also for additional filtering and other signal processing techniques, beyond the standard algorithms used in ECG measurement, to avoid misinterpreting the ST segment as a separate heart beat in some of the new lead cases.

One example of a suitable signal processing technique to isolate and identify the QRS complex, and to differentiate it from a potentially uncharacteristically high ST segment, would revolve around the relative constant duration of the QRS complex for the population at large. This duration is typically between 60 and 70 milliseconds for a normal healthy subject. This corresponds to a frequency between 15 and 20 Hertz. A band pass filter with center frequency at or near 17 Hertz can therefore be useful in filtering out ST segment energy, along with other unwanted noise, while allowing QRS complex energy to pass through. This method eliminates all other information present in the cardiac waveform, along with the noise, leaving only the QRS complex. As applied herein, the QRS complex is the only element of the signal that is required.

Well known to those of ordinary skill in the art, signal processing and filtering techniques can improve results further. The same wires used to deliver the music signal to the headphones may in some cases be used to carry the heart rate signal to the heart rate detection circuitry. A good filtering method for this case follows from the description of the heart rate energy, and especially the QRS complex which is the most detectable element of a heart beat signature. One possible first step is to filter the music signal delivered from the audio source device, eliminating all energy in and around 17 Hz. This can be done with a notch filter, for example, with center frequency of 17 Hz. Music or other audio lacking this extremely low frequency energy can be delivered to the listener without any significant loss of quality. The placement of the conductors on or around the ears results in an addition of energy from the heart rate, which is at very low amplitude and a very low frequency, and which does not affect the music quality. At the heart rate detection circuitry, all energy except that at or near 17 Hz is filtered out using, for example, a band pass filter with center frequency of 17 Hz. This subtracts the music and any other noise artifacts, leaving only the QRS portion of the heart beat energy, which can then be passed on to the remainder of the heart beat detection circuitry. Other standard techniques may be useful. One example of such is inverting the audio signal and adding it to the detected signal, prior to heart rate detection, thus effectively subtracting the audio energy from the signal.

### Block Diagrams and Logic Flow

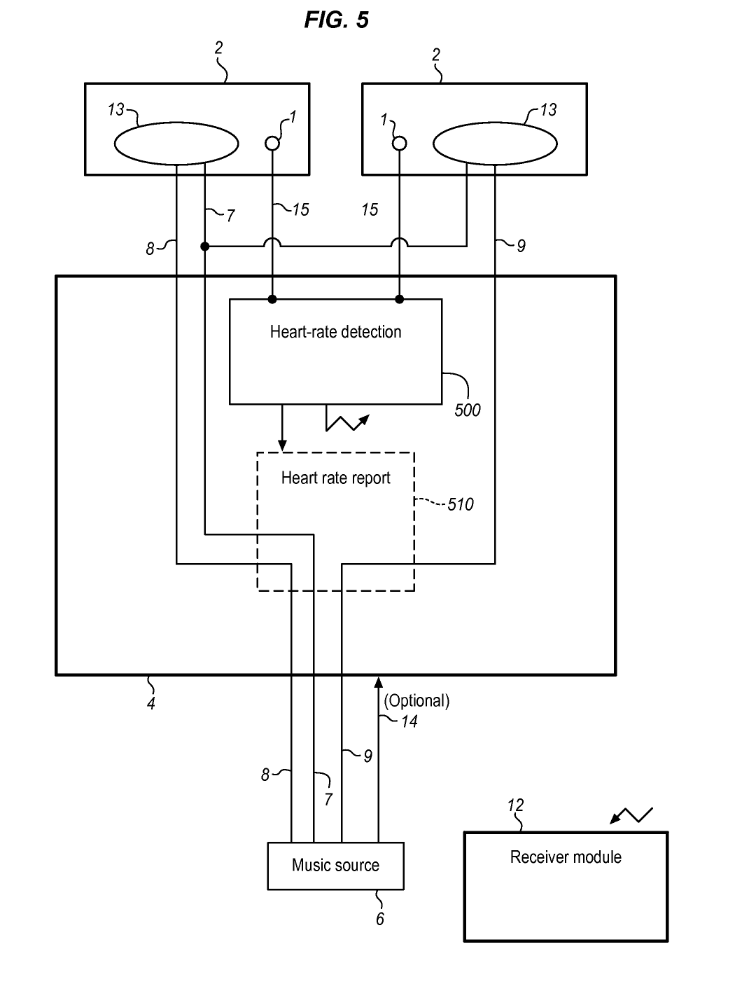

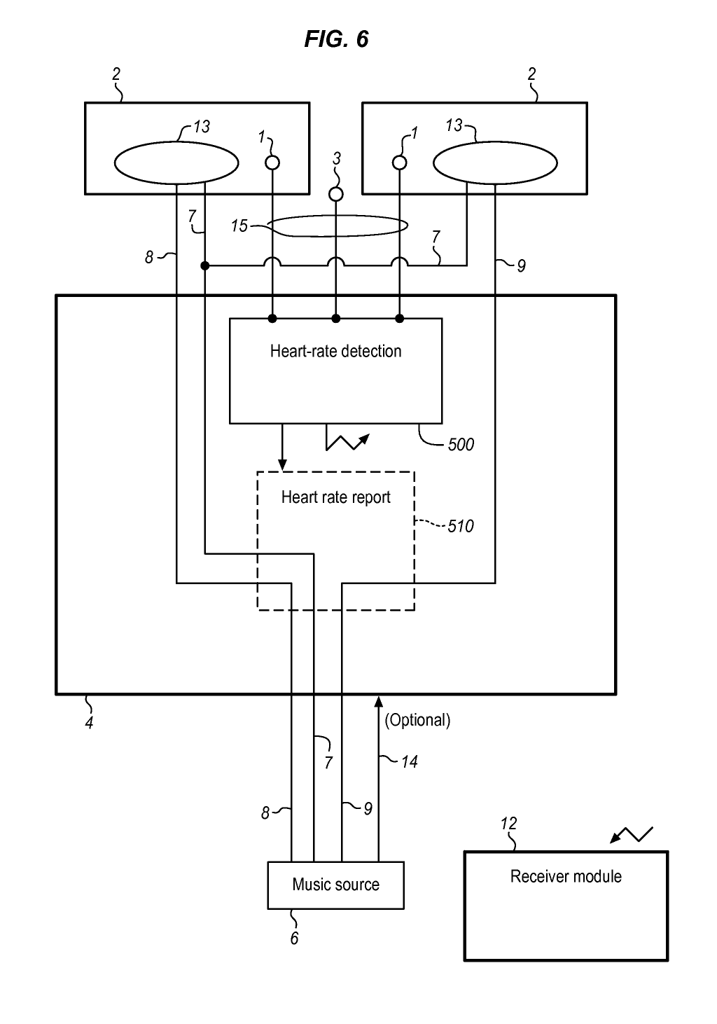

Details of exemplary connections coupling the various components of an exemplary system are shown in FIGS. 5 and 6. FIG. 5 shows a system with two electrodes 1 each coupled to a corresponding headphone speaker 13. Audio signals from the audio source device 6 are passed through the heart rate detection module 4 via conductors 7, 8, and 9 (common ground, left channel, and right channel, respectively—collectively referred to above as wires 5). Module 4 includes detection circuit 500 coupled to the electrodes 1 via wires 15. As noted above, wires 15 may be integral with wires 7-9 (collectively wires 5) such that the heart beat signals may be superposed on the same wires as the audio signals. Circuit 500 may therefore include appropriate filtering and processing to extract hear beat signals superposed on the same conductors as audio signals. Heart rate report element 510 (also referred to herein as a “presentation device”) receives signals representing the current heart rate determined by circuit 500 and presents the heart rate information to the wearer as audio signals applied to conductors 7-9. In particular, for example, element 510 may interrupt the transmission of audio signals on wires 7-9 and apply (inject) generated audio signals informing the wearer of the current heart rate. In addition or in the alternative, a presentation device 12 may be worn and may receive the heart rate information from circuit 500. Presentation device 12 may be coupled to circuit 500 by wired or wireless means. Further, presentation device 12 may include a display device (not shown) to present the heart rate information to the wearer as displayed text or graphics (i.e., visual information). The presentation device 12 may also include a memory (not shown) to store the information for later processing and presentation. Thus presentation device 12 may also include an interface (not shown) to external elements such as a computer with which the presentation device 12 may exchange stored data for later processing and presentation. Still further, presentation device 12 may be physically integral with module 4 or may be separate and physically distinct worn in another location on the user of the system. Still further, power required to operate module 4 may be supplied by any suitable internal or external source. For example, audio source device 6 may provide power via coupling 14 to operate module 4. In the alternative, module 4 may incorporate a battery or other suitable integral power sources. FIG. 6 shows a system identical to that of FIG. 5 with the addition of a third electrode 3.

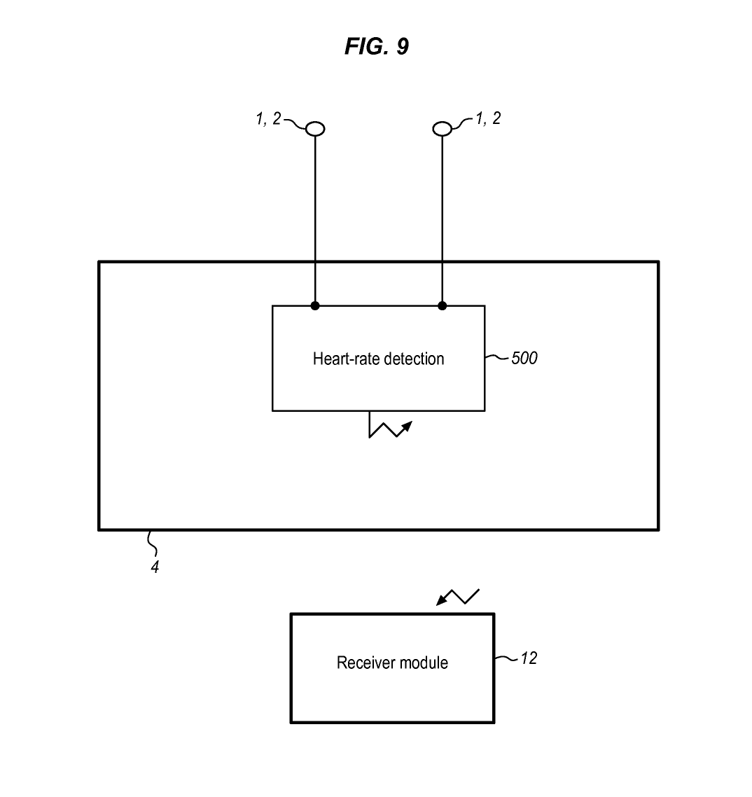

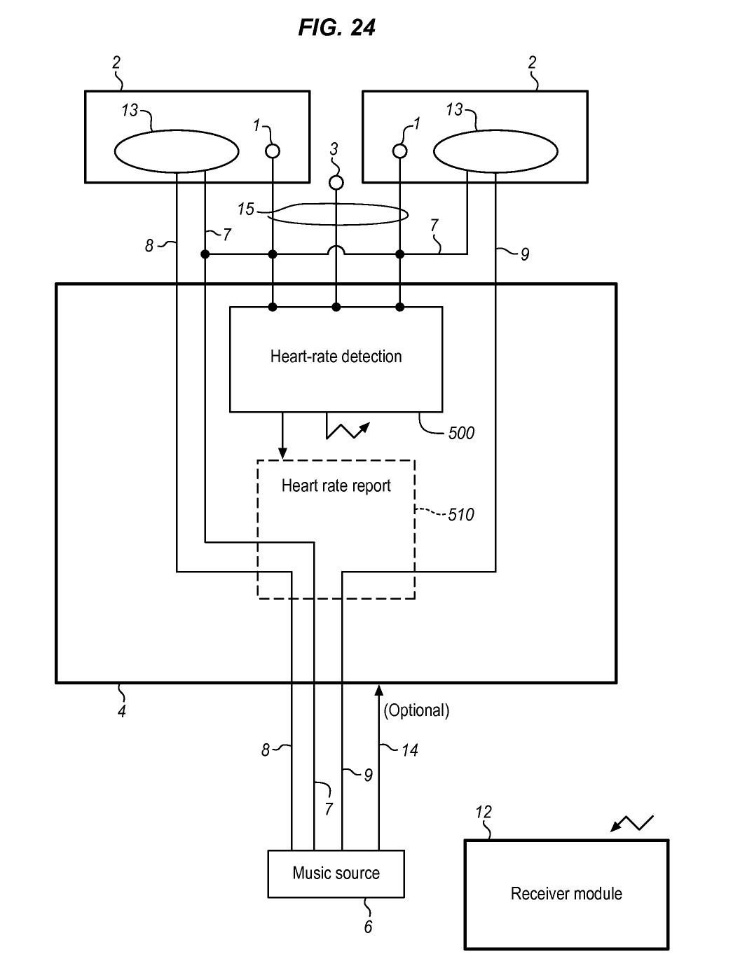

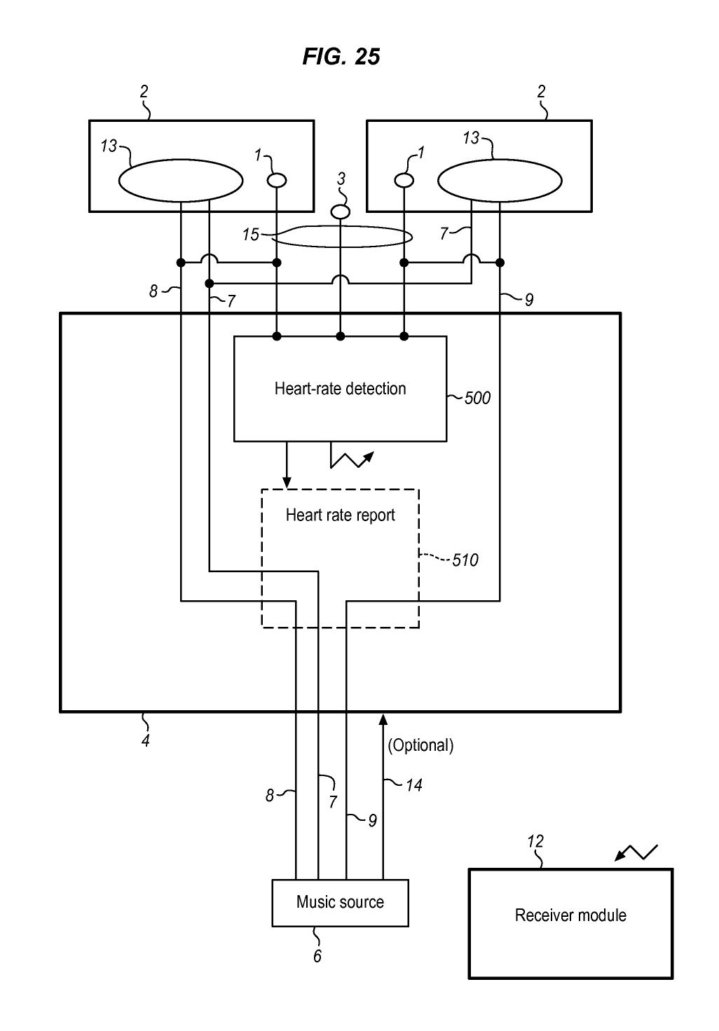

FIGS. 24 and 25 show two exemplary variations of the system of FIG. 6 in which the wires that connect an audio source device to headphones are the same wires that connect the electrodes to the heart rate detection circuitry. In FIG. 24, the ground wire which is connected to both sides of the headphones is also the wire connected to the electrodes. In FIG. 25, the signal wires audio left and audio right, are employed to connect a left electrode and right electrode, respectively, to the heart beat detection circuitry. FIG. 9 shows a simplified embodiment where the heart rate detection module 4 is independent of any audio source device. Detected heart rate information is simply forwarded to the presentation device 12 for presentation to a wearer.

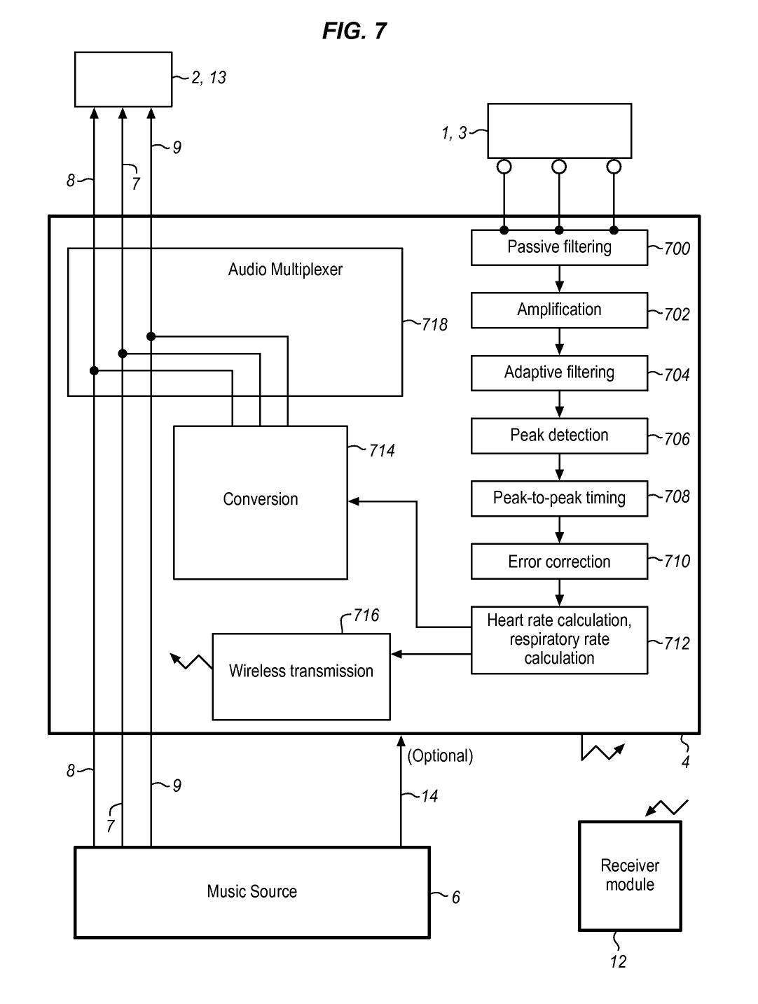

Further exemplary details of the operations within the heart rate detection module 4 are shown in FIG. 7. The detection circuit measures changes in the electrical voltage potential between the conductors (1 and optionally 3) depending on the specific leads utilized. It passes this measured signal through various analytical steps which may include passive filtering 700, amplification 702, adaptive filtering 704, peak detection 706, peak-to-peak timing 708, error correction 710, and calculations 712 (e.g., with averaging to smooth the data generated). The outputs of this module 712 include a continuously updated heart rate value, and optionally a continuously updated respiratory rate value. This information may be applied to an audio conversion element 714 to generate an audio signal applied via module 718 to the speakers 13 in headphones 2 via conductors 7, 8, and 9. The audio signal may be simple beeps or may be generated speech periodically announcing in numbers the current hear rate. In addition the output of module 712 may also (or alternatively) be applied to wireless transmitter 716 for transmission of information to a receiver module 12. The receiver 12 may present the information as displayed graphics and/or text.

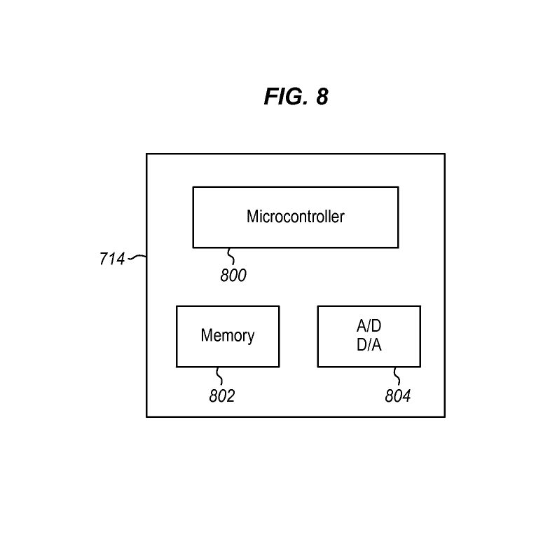

FIG. 8 shows a block diagram of exemplary components in the audio conversion element 714 of FIG. 7. Conversion element 714 may include a microcontroller 800 and memory 802 for storing information as received and as converted to text, graphics, and/or audio signals. An analog to digital and digital to analog (ADC and DAC) element 804 may be used to convert the received heart rate and other information, if needed, from an analog signal to digital information and to convert digital information to an audio signal for injection on the speakers of attached headphones.

The continuously updated information which is the output of the detection and analysis circuits may be presented to a user in several ways as noted above, In particular, for example, the information may be converted to an audio signal as shown in FIG. 3 such as the spoken number representing the current heart rate and periodically interspersed with the audio signal. In addition or in the alternative, the spoken information may be added to the audio signal as a voice-over rather than replacing the music or other information from the audio source. Further, for example, the heart rate information can be converted to any other audio signal such as a tone or periodic beeps varying in duration, frequency, or period, in a manner to represent varying heart rates to the listener (e.g., heart rate low or high threshold values reached). Also, it can be used to modify the audio input from an audio source device, for example by making the music relatively quieter as the user's heart rate or respiratory rate decreases.

The heart rate and/or respiratory rate information can also be passed via a wireless transmitter or through a wired connection which communicates to a separate monitoring receiver module, such as a wristwatch, handlebar-mounted display, mobile telephone or other monitoring device. In another embodiment, the heart rate/respiratory rate information can be communicated, wirelessly or via wires, to the audio source device for display, storage or forwarding by that device, provided that the audio source device contains features required to enable this.

In another embodiment, the audio source device and/or telecommunications device may be designed with the intent of offering heart rate measurement as one feature. In this embodiment, the circuitry shown in heart rate detection module would be contained within the audio source device. In similar fashion, in an embodiment of this type, operations required for the reporting of heart rate information to the user would also be integrated into the audio source device, rather than in a separate module.

What is claimed is:

1. An apparatus for detecting heart rate of a user, the apparatus comprising:
a first electrode adapted to be placed in contact with the user's head;
a second electrode adapted to be placed in contact with the user's skin;
a heart rate detection circuit electrically coupled to the first and second electrodes and adapted to determine the heart rate of the user from electrical signals sensed from the first and second electrodes; and
a presentation device communicatively coupled to the heart rate detection circuit to receive heart rate information regarding the heart rate from the heart rate detection circuit and adapted to present the heart rate information to the user.

2. The apparatus of claim 1
wherein the first electrode is adapted to be positioned in contact with the user's skin in, on, and/or around the ear.

3. The apparatus of claim 1
wherein the first electrode is adapted to be positioned in contact with the user's skin in, on, and/or around the temple.

4. The apparatus of claim 1
wherein the first electrode is integral with headwear apparel.

5. The apparatus of claim 4 wherein the headwear apparel includes any of: headphones, eyeglasses, sunglasses, hats, helmets, headbands, goggles, goggle straps, eyeglass or sunglass lanyards, and/or ear muffs.

6. The apparatus of claim 1
wherein the heart rate detection circuit is further adapted to determine other physiological data of the user from electrical signals sensed from the first and second electrodes, wherein the other physiological data is derived from the heart rate information; and
wherein the presentation device is further adapted to receive the other physiological data from the heart rate detection circuit and to present the other physiological information to the user.

7. The apparatus of claim 6
wherein the other physiological data is a respiration rate of the user.

8. The apparatus of claim 1
wherein the presentation device is coupled to the heart rate detection device by a wireless interface.

9. The apparatus of claim 1
wherein the presentation device presents the heart rate information to the user as displayed information.

10. The apparatus of claim 1
wherein the first electrode is integral with a speaker.

11. The apparatus of claim 10
wherein the presentation device presents the heart rate information to the user as audio signals applied to the speaker.

12. The apparatus of claim 10 further comprising:
an audio source device coupled to the speaker for applying audio signals to the speaker.

13. The apparatus of claim 12
wherein the presentation device is coupled to the audio source device such that the heart rate information is presented to the user as audio signals applied to the speaker.

14. The apparatus of claim 1
wherein the second electrode is in contact with the user's head.

15. The apparatus of claim 14
wherein the second electrode is adapted to be positioned in contact with the user's skin in, on, and/or around the ear.

16. The apparatus of claim 14
wherein the second electrode is adapted to be positioned in contact with the user's skin in, on, and/or around the temple.

17. The apparatus of claim 14
wherein the first electrode is integral with headwear apparel.

18. The apparatus of claim 17 wherein the headwear apparel includes any of: headphones, eyeglasses, sunglasses, hats, helmets, headbands, goggles, goggle straps, eyeglass or sunglass lanyards, and/or ear muffs.

19. The apparatus of claim 1 further comprising:
a third electrode positioned in contact with the user's skin other than on the user's head,
wherein the heart rate detection circuit is further adapted to determine the heart rate from electrical signals sensed from the first, second, and third electrodes.

20. The apparatus of claim 19
wherein the third electrode is positioned on the user's arm.

21. The apparatus of claim 19
wherein the third electrode is positioned on the user's wrist.

22. The apparatus of claim 19
wherein the third electrode is positioned on the user's waist.

23. A system comprising:
a speaker positioned proximate an ear of a user;
a first electrode integral with the speaker and positioned such that the first electrode is in contact with the skin of said ear;
a second electrode positioned in contact with the user's skin;
an audio source device coupled for applying audio signals to the speaker to generate sound audible to the user; and
a heart rate detection circuit coupled to the first and second electrodes and adapted to determine the heart rate of the user from electrical signals sensed from the first and second electrodes wherein the heart rate detection circuit is adapted to generate heart rate information for presentation to the user.

24. The system of claim 23
wherein the heart rate detection circuit is coupled to the speaker, and
wherein the heart rate detection circuit is adapted to generate heart rate related audio signals representing the heart rate information and to apply the generated heart rate related audio signals to the speaker.

25. The system of claim 23 further comprising:
a presentation device communicatively coupled to the heart rate detection circuit to receive the heart rate information and adapted to present the heart rate information to the user.

26. The system of claim 25
wherein the presentation device further comprises a display device for displaying to the user visual information representing the heart rate information.

27. The system of claim 25
wherein the presentation device is coupled to the heart rate detection circuit by a wireless interface.

28. The system of claim 23 further comprising:
a third electrode positioned in the user's skin other than on the user's head,
wherein the heart rate detection circuit is further adapted to determine the heart rate from electrical signals sensed from the first, second, and third electrodes.

29. The system of claim 28 wherein the third electrode is integral to a mounting device for holding or carrying an audio source device while exercising.

30. The system of claim 23 further comprising:
a second speaker positioned proximate the other ear of said user wherein the second speaker is coupled to receive audio signals from the audio source device,
wherein the second electrode is integral with the second speaker.

31. The system of claim 23 wherein the second electrode is positioned on the user's arm.

32. The system of claim 23 wherein the second electrode is positioned on the user's wrist.

33. The system of claim 23 wherein the second electrode is positioned on the user's torso.

34. The system of claim 23 wherein the audio source device comprises a music player.

35. The system of claim 23 wherein the audio source device comprises a mobile telephone.
